(19) United States
(12) Patent Application Publication
(10) Pub. No.: US 2022/0267774 A1
(43) Pub. Date: Aug. 25, 2022

(54) METHODS AND COMPOSITIONS TO DIRECT BREAKDOWN OF INSULIN MRNA IN BENIGN FASHION

(71) Applicant: CITY OF HOPE, Duarte, CA (US)

(72) Inventors: Bart Otto ROEP, Duarte, CA (US); Rene Matthijs VAN TIENHOVEN, Duarte, CA (US)

(73) Assignee: CITY OF HOPE, Duarte, CA (US)

(21) Appl. No.: 17/692,048

(22) Filed: Mar. 10, 2022

Related U.S. Application Data
(63) Continuation of application No. PCT/US20/50572, filed on Sep. 11, 2020.
(60) Provisional application No. 62/899,065, filed on Sep. 11, 2019.
(60) Provisional application No. 63/047,182, filed on Jul. 1, 2020.

(51) Int. Cl. C12N 15/113; A61P 5/48; A61K 35/39

(52) U.S. Cl. CPC C12N 15/1136; A61P 5/48; A61K 35/39

(57) ABSTRACT

Methods and compositions discussed herein allow for preventing or treating type 1 diabetes (T1D) including directing the breakdown of insulin mRNA.

### PRIORITY CLAIM

This application is a continuation of International Patent Application No. PCT/US2020/050572, filed Sep. 11, 2020, which claims priority to U.S. Provisional Application No. 62/899,065, filed Sep. 11, 2019 and U.S. Provisional Application No. 63/047,182, filed Jul. 1, 2020, all of which are incorporated herein by reference in their entirety.

### SEQUENCE LISTING

This application contains a Sequence Listing, which was submitted in ASCII format via EFS-Web, and is hereby incorporated by reference in its entirety. The ASCII copy, created on Feb. 25, 2022, is named SequenceListing.txt and is 8 KB in size.

### FIELD OF THE INVENTION

The invention relates to compositions and methods to direct the breakdown of insulin mRNA in a benign fashion and applications thereof.

### BACKGROUND

Identification of epitopes that are recognized by diabetogenic T cells and cause selective beta cell destruction in type 1 diabetes (T1D) has focused on peptides originating from native beta cell proteins. Translational errors represent a major potential source of antigenic peptides to which central immune tolerance is lacking. For example, an alternative open reading frame(ORF)within human insulin mRNA has been described, where that alternative ORF encodes a highly immunogenic polypeptide that is targeted by T cells in T1D patients. Studies have shown that cytotoxic T cells directed against the N-terminal peptide of this nonconventional product are present in the circulation of individuals diagnosed with T1D and provide direct evidence that such CD8⁺ T cells are capable of killing human beta cells and thereby may be diabetogenic. Those studies revealed a new source of nonconventional polypeptides that act as self-epitopes in clinical autoimmune disease.

Islet-reactive CD8⁺ T cells selectively and progressively destroy the insulin-producing beta cells in T1D. Metabolic or inflammatory stress in the vicinity of the beta cells may contribute to the generation of neoantigens to which central immune tolerance is absent, thereby triggering autoimmunity. For example, larger numbers of splicing events are detected in human beta cells maintained in vitro in the presence of proinflammatory cytokines, mimicking the pathophysiological conditions of T1D. In addition, CD4⁺ T cells that recognize deamidated autoantigens or fusion epitopes have been detected in individuals with T1D. Inflammatory stress may perturb the cellular equilibrium and affect high-fidelity transcriptional and translational processes during conversion of the genetic information into proteins. In tumors, uncontrolled cell proliferation correlates with enhanced translation and accumulation of aberrant translation products. Such so-called defective ribosomal products (DRiPs), arising from the translation of normally untranslated regions (UTR), ribosomal frame-shifting or alternative initiation of translation, generate a unique class of tumor-associated antigens that are selectively expressed by malignant cells.

In a process similar to the emergence of DRiPs during tumor development, the high demand for insulin may lead to the generation of aberrant insulin polypeptides by impaired translation fidelity, rendering beta cells immunogenic. As such, it would be beneficial to develop therapeutic intervention to target and reduce DRiP production to reduce immunogenicity and to prevent or ameliorate the development of T1D.

### SUMMARY

Methods and compositions discussed herein allow for preventing or treating type 1 diabetes (T1D) including directing the breakdown of insulin mRNA.

Methods and compositions discussed herein allow for inhibiting defective ribosomal product (DRiP) expression, including using gene editing, e.g., using CRISPR-Cas9 mediated methods to replace a to replace a single nucleotide polymorphism (SNP) in the 3′ untranslated region (UTR) of the insulin (INS) gene.

In an embodiment, a targeting molecule for inhibiting DRiP expression includes a donor template comprising a nucleic acid sequence that encodes an mRNA sequence comprising CUGCAG (SEQ ID NO:1) to replace a SNP in the 3′ untranslated region of the INS gene.

In an embodiment, a targeting molecule for inhibiting DRiP expression includes a donor template comprising a nucleic acid sequence that encodes an mRNA sequence comprising CUGCAG (SEQ ID NO:1) to replace a SNP in the 3′ untranslated region of the INS gene wherein the SNP is rs3842752.

In an embodiment, targeting molecule for inhibiting defective ribosomal product DRiP expression comprises (i) a donor template comprising a nucleic acid sequence that encodes an mRNA sequence comprising CUGCAG (SEQ ID NO:1) to replace a SNP in the 3′ untranslated region of the insulin (INS) gene; (ii) a nucleic acid sequence that is fully or partially complementary to at least a portion of INS mRNA; or (iii) an antibody or fragment thereof that targets; wherein the targeting molecule inhibits the expression of DRiP. In certain embodiments, the nucleic acid sequence that is fully or partially complementary to at least a portion of INS mRNA comprises a nucleic acid sequence beginning at AUG341 in the INS gene. In certain embodiments, the nucleic acid sequence that is fully or partially complementary to at least a portion of INS mRNA comprises a nucleic acid sequence beginning at AUG₆₀ in the INS gene. In certain embodiments, the nucleic acid sequence that is fully or partially complementary to at least a portion of INS mRNA comprises a nucleic acid sequence beginning at AUG72 in the INS gene.

In an embodiment, a pharmaceutical composition comprising a targeting molecule disclosed herein and one or more pharmaceutically acceptable carriers or excipients is provided.

In an embodiment, a method for preventing or T1D is provided, comprising administering to a subject a therapeutically effective amount of the targeting molecule as disclosed herein, wherein the subject is identified as having a susceptible SNP mutation or lacking a protective SNP mutation.

In an embodiment, a method for inhibiting the expression or activity of DRiPs in pancreatic beta cells comprising contacting the pancreatic beta cells with an effective amount of a targeting molecule as disclosed herein, wherein the pancreatic beta cells have a susceptible SNP mutation or lack a protective SNP mutation. In certain embodiments, the SNP is rs3842752. A susceptible SNP mutation in rs3842752 includes a C/C at position 401, whereas a protective SNP mutation in rs3842752 includes a U/A at position 401. The nucleic acid sequence as disclosed herein may be any suitable nucleic acid sequence including, for example, an antisense oligo or a cDNA molecule.

### BRIEF DESCRIPTION OF THE DRAWINGS

This application contains at least one drawing executed in color. Copies of this application with color drawing(s) will be provided by the Office upon request and payment of the necessary fees.

FIG. 1 is a schematic depicting DRiP and SPLICE formation. The bona fide preproinsulin (PPI) translation initiation site is indicated as AUG₆₀ and an alternative downstream translation initiation site is indicated as AUG341. SNPs rs3842752 and rs3842753 are indicated by red bands. T cells are reactive to an epitope that corresponds to the first 9 amino acids (INS-DRiP₁₋₉ epitope, MLYQHLLPL(SEQ ID NO:2)) of DRiP indicated as a darker yellow band in the Figure above (“immunoreactive portion”). This immunoreactive portion is not present in SPLICE.

FIGS. 2A-D depict immunostaining illustrating SPLICE is expressed in delta cells. FIG. 2A is INS staining (green), FIG. 2B is SPLICE staining (yellow), FIG. 2C is SS staining (red), and FIG. 2D is a merged image of FIGS. 2A-C and including Hoechst staining (blue).

FIG. 3 depicts immunostaining illustrating that DRiP is heterogeneously expressed over the pancreas and colocalizes with insulin in beta cells. Staining is pictured from a DRiP positive islet (upper panels) and a DRiP negative islet (lower panels). INS staining is visualized in green, DRiP staining is visualized in red, SS staining is visualized in purple, and Hoechst staining is visualized in blue.

FIG. 4 is a chart depicting genetic risk for T1D. Bars represent associated loci from genome-wide association (GWA) studies. HLA represents the highest genetic risk and an INS gene mutation represents the second-highest genetic risk.

FIGS. 5A-C includes a schematic diagram of the INS gene including the VNTR promotor region (top panel, A), and panels B and C (below).

FIG. 6 includes a chart illustrating that longer VNTR is suspected of causing a central tolerance to proinsulin.

FIG. 7 depicts charts illustrating lymphocyte proliferation against the recombinant INS-DRiP variants (C-H and R-P) in peripheral blood mononuclear cells (PBMCs) from fresh blood of individuals with T1D who are homozygous for the susceptible INS-SNP phenotype R-P/R-P (left) or heterozygous with the protective phenotype HLA-DQ6.2 or INS-SNP C-H (right). The experimental results depicted in this Figure indicate that patients with the INS-SNP phenotype R-P/R-P (left) react to both recombinant INS-DRiP variants (C-H and R-P).

FIGS. 8A-B depicts experimental results analyzing the effect of ER stress on translation initiation indicating that DRiP expression is increased during ER stress. FIG. 8A is a western blot of 293T cell lysates (left to right) in nontransfected cells (NT), or cells transfected with INS-DRiP-GFP that were untreated (−), stimulated with thapsigargin (TG), or stimulated with tunicamycin (TM). The western blot in FIG. 8A is labeled using anti-GFP, anti-insulin, and anti-actin antibodies. FIG. 8B is a chart depicting densitometry analyses of the western blot in FIG. 8A analyzing the GFP (black bars) and insulin bands (gray bars) and shown as induction ratio±s.d. where the intensity of the nontreated cells was used as reference (right).

FIG. 9 is a schematic diagram depicting the unstressed endoplasmic reticulum.

FIG. 10 is a schematic diagram depicting the unfolded protein response (UPR).

FIG. 11 is a schematic diagram depicting the role of IRE1 in UPR.

FIG. 12 is a schematic representation of open reading frames (ORFs) found in the human insulin mRNA (SEQ ID NOS:3-5). Full-length insulin mRNA with the bona fide PPI ORF (black uppercase letters), 5′ and 3′ UTRs (gray uppercase letters) and the poly(A) signal sequence (bold gray letters) are shown. The PPI amino acid sequence is shown in blue font, the amino acid sequence of the +2 reading frame is shown in small, light gray font and the amino acid sequence of the alternative open reading frame (altORF) (INS-DRiP) is depicted below the mRNA sequence in bold red. All AUG codons within the mRNA are framed with a black box, and those used as translation initiation site are indicated with colors corresponding to the resulting amino acid sequence. The * indicate the stop codons in the given amino acid sequence. The putative non-AUG (CUG) start site upstream of the DRiP sequence is framed with red dashed line. The 3′-UTR SNPs are annotated and both polymorphisms are depicted, as are the potentially affected amino acids in the nonconventional polypeptide.

FIGS. 13A-C is a series of illustrations depicting IRE1α specificity. FIG. 13A depicts hairpin structures (SEQ ID NOS:6-14). FIG. 13B depicts XBP1 mRNA splicing (blue) and RIDD of SPARC mRNA (red). FIG. 13C shows the consensus sequence of the protective rs3842752 SNP (SEQ ID NO:1).

FIGS. 14A-B depicts schematic diagrams of INS/DRiP mRNA having the protective SNP (U/A) (FIG. 14A, green) and the susceptible SNP (C/C) (FIG. 14B, red) and IRE1.

FIG. 15 is a schematic showing the experimental design for evaluating a subject's rs3842752 SNP status according to one embodiment.

FIG. 16 is schematic showing the experimental design for evaluating a subject's rs3842752 SNP status according to another embodiment.

FIG. 17 is a Northern blot showing results from an experiment performed according to the design of FIG. 15 for two donors, subject Hu 1087 and Hu 1000.

FIGS. 18A-B illustrates cleavage of in vitro transcribed RNA by IRE1. FIG. 18A shows results of an in vitro cleavage assay where lug of RNA was incubated with 0.5ug IRE1 (+) or without IRE1 (−) for 30min. After the in vitro cleavage assay, IRE1 was heat inactivated and 10Ong of RNA cleave product was resolved on a 10% TBE-UREA gel. FIG. 18B shows results of quantification of the RNA cleave products, where the band intensity of whole (uncleaved) RNA products were analyzed for incubation with and without IRE1 and plotted as % cleaved compared to the IRE negative (−) bands.

FIG. 19 is a flowchart illustrating the processing of donor islets.

FIGS. 20A-C illustrates quantification of in vitro cleavage of insulin mRNA from human donor islet cells. FIG. 20A shows results of a Northern blot of INS mRNA with and without IRE1. FIG. 20B is a pie chart showing the quantification of band intensity for protective donor RNA shown in FIG. 20A. FIG. 20C is a pie chart showing the quantification of band intensity for susceptible donor RNA shown in FIG. 20A.

FIG. 21 is a graph showing stemloop cleavage by IRE1 in susceptible (top line, CCGCAG, SEQ ID NO:15) and protective (bottom line, CUGCAG, SEQ ID NO:1) insulin variants.

FIGS. 22A-B illustrates ex vivo cleavage of insulin mRNA in a cell line. FIG. 22A is a bar graph illustrating insulin mRNA expression change when beta cells are stressed with Thapsigargin (TG). FIG. 22B is a bar graph illustrating DRiP protein expression when beta cells are stressed with TG.

FIGS. 23A-D shows the structure of the susceptible and protective insulin mRNA and its relative rate of decay by IRE1a. FIG. 23A shows the structure of the protective insulin mRNA containing the CUGCAG-motif (SEQ ID NO:16) and hairpin structure (SEQ ID NO:17), whereas FIG. 23B shows the structure of the susceptible mRNA containing the CCGCAG motif without hairpin structure (SEQ ID NOS:18-19). FIG. 23C is a graph showing luciferase activity in protective Rluc-INS transfected cells (blue) compared to susceptible Rluc-INS transfected cells (red), both treated with increasing amounts of thapsigargin (TG). FIG. 23D is a graph showing cleavage of human heterozygous protective insulin mRNA (dark blue, n=2, homozygous protective (light blue, n=1), and homozygous susceptible islet donors (red, n=2) by activated IRE1a.

FIG. 24 shows a schematic of vectors carrying protective Rluc-INS (top) or susceptible Rluc-INS (bottom).

FIG. 25 is RESERVED

FIG. 26 is RESERVED

FIGS. 27A-I illustrates the better vitality and function of human islets carrying the protective INS variant. FIG. 27A is a graph showing oxygen consumption rate (OCR) over time in islets with the protective INS variant (blue) and in islets carrying the homozygous susceptible INS variant (red). FIG. 27B is a bar graph showing the fold change in (OCR) in islets with the protective INS variant (blue) and in islets carrying the homozygous susceptible INS variant (red). FIG. 27C is a graph showing insulin secretion by islets with the protective INS variant at low glucose as compared to high glucose levels. FIG. 27D is a graph showing insulin secretion by islets with the susceptible INS variant at low glucose as compared to high glucose levels. FIG. 27E is a bar graph comparing insulin secretion between islets with the susceptible INS variant (red) and islets with the protective INS variant (blue) at high glucose levels and low glucose levels. FIG. 27F is a graph showing blood glucose levels in STZ-treated diabetic NOD SCID mice transplanted with human islets carrying the protective INS variant. FIG. 27G is a graph showing blood glucose levels in STZ-treated diabetic NOD SCID mice transplanted with human islets carrying the susceptible INS variant. FIG. 27H is a bar graph comparing the time to reverse diabetes (i.e., became normoglycemic) in diabetes-reversed NOD SCID mice transplanted with human islets carrying the protective INS variant (blue, n=14) or the susceptible INS variant (red, n=10). FIG. 271 is a graph showing the speed of effectiveness (as measured by % diabetes reversal) of treatment of diabetic NOD SCID mice transplanted with human islets carrying the protective INS variant (blue) or the susceptible INS variant (red).

FIG. 28 is a schematic shoeing an INS-DRiP-GFP construct according to some embodiments. Blue lines represent the two SNPs.

FIG. 29 shows DRiP-GFP expression of the protective vs susceptible construct. GFP expression is shown for no treatment (CTRL) (left panel), and for 2 mM thapsigargin treatment (TG) (right panel).

FIG. 30 is a graph showing the Median Fluorescent Intensity (MFI) of the DRiP-GFP positive population represented in FIG. 29.

### DETAILED DESCRIPTION

### Type 1 Diabetes (T1D)

During type 1 diabetes (T1D) development, cells in the islets of Langerhans engage adaptive mechanisms in response to inflammatory signals to cope with stress, to restore cellular homeostasis, and to preserve cell function. Islet-reactive CD8⁺ T cells selectively and progressively destroy the insulin-producing beta cells in patients with T1D.

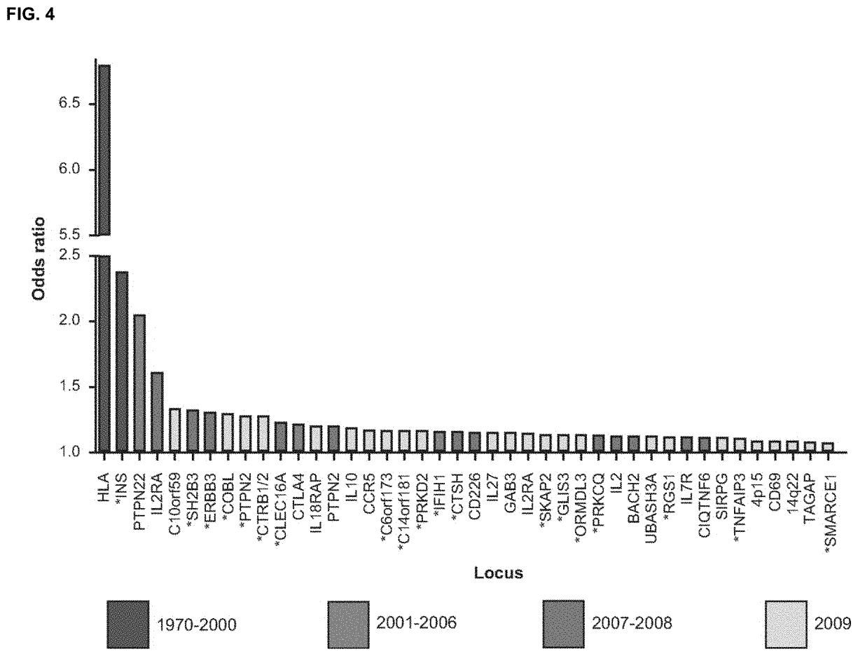

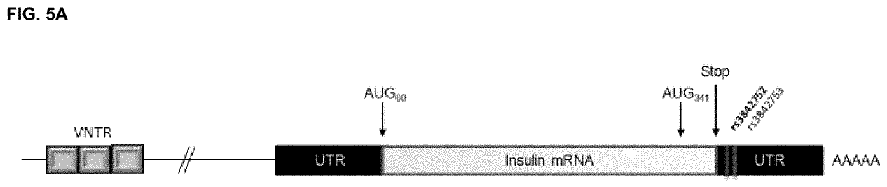

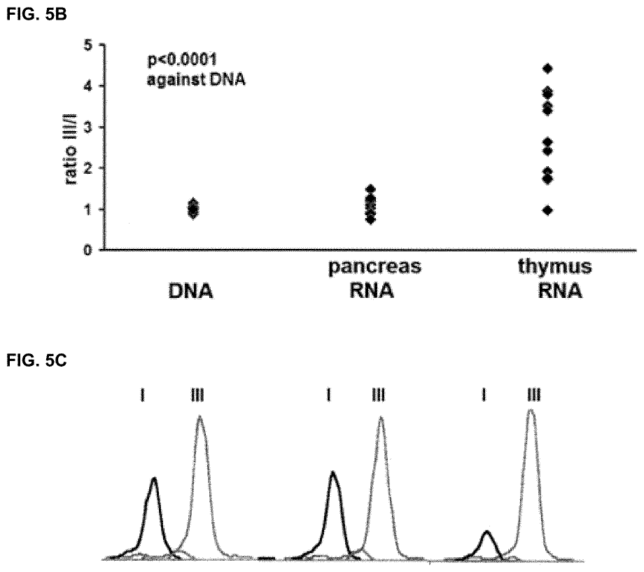

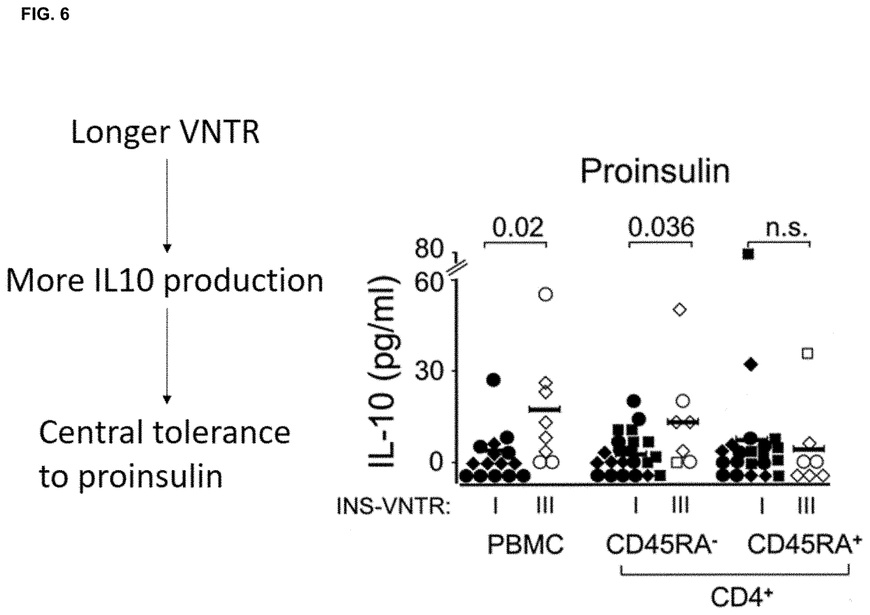

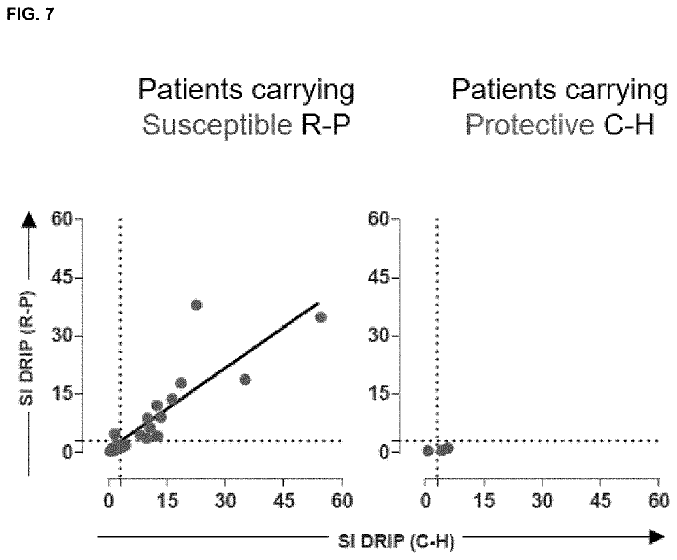

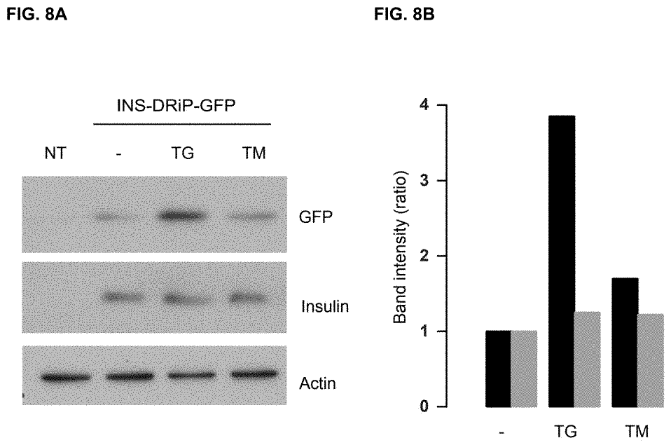

Two single nucleotides polymorphisms (SNPs) in the 3′ untranslated region (UTR) of the insulin (INS) gene are the second strongest genetic risk markers for development of T1D. See FIG. 4. The ‘classic’ explanation for this phenomenon relates to the rate of INS transcription and translation in the thymus versus pancreatic islets. The protective SNPs, which are in 100% linkage disequilibrium with INS promoter polymorphisms defined the VNTR length, cause expression of proinsulin in the thymus. See FIG. 5. This results in so-called ‘thymic education’ or ‘central tolerance,’ as T cells reactive with proteins in the thymus will be eliminated in their development. Thus, T cells to insulin are absent in genetically protected individuals. Stressed beta cells are prone to produce alternative insulin mRNA products, including defective ribosomal products (DRiPs) (Kracht, Nature Medicine 2017) and splice products that are uniquely expressed in islets, and unlikely to be expressed in the thymus.

Uncontrolled cell proliferation in tumor cells correlates with enhanced translation and accumulation of aberrant translation products. DRiPs arise from the translation of normally untranslated UTRs, ribosomal frame-shifting, or alternative initiation of translation and generate a unique class of tumor-associated antigens that are selectively expressed by malignant cells. Human pancreatic beta cells are insulin factories dedicated to the maintenance of glucose homeostasis; insulin, stored in secretory granules, represents 10-15% of the protein content of these cells. Upon glucose challenge, insulin molecules are released into the circulation by exocytosis, and insulin mRNA is rapidly translated by polysomes to increase insulin biosynthesis. Studies of samples from humans with T1D and mouse models of the disease indicate that native insulin and its precursors act as primary autoantigens, and fragments of the signal peptide of the preproinsulin (PPI) were identified as main targets of cytotoxic islet-autoreactive CD8⁺ T cells in human T1D. In a process similar to the emergence of DRiPs during tumor development, the high demand for insulin may lead to the generation of aberrant insulin polypeptides by impaired translation fidelity, rendering beta cells immunogenic. These errors may yield antigens that trigger or accelerate islet autoimmunity in T1D, particularly as a result of stresses that, for example, may be evoked by viral infection, reduced pancreas size or inflammation.

Individuals carrying the protective INS SNPs do not respond to DRiP proteins either, even the few patients that in spite of carrying protective INS SNPs developed T1D. It was discovered that certain proteins activated under stress called IRE1α recognize sequences in the UTR of mRNA leading the cuts in this RNA and accelerated breakdown, prevention further production of unfolded proteins that could contribute to ER stress and subsequent cell death. Strikingly, the binding and cutting motif of IRE1α covers the protective SNP, and is absent in the susceptible SNP, preventing IRE1α to cut and break down proinsulin mRNA in carriers of the susceptible INS SNP. Consequently, carrier of the susceptible SNP are still able to produce INS stress proteins such as DRiP, whereas individuals protected by the INS SNP break down INS mRNA and are less capable to produce DRiPs and develop autoimmune responses against these error proteins. This mechanism may explain why individuals with protective SNPs do not respond to DRiP and rarely develop T1D, whereas those with susceptible INS SNPs produce DRiP in their stressed beta cells and can develop pathogenic autoimmunity against this DRiP error proteins, leading to T1D. This alternative mechanism of genetic protection inferred by INS SNPs may act in concert with this established mechanism of central tolerance See Example 1 below.

Methods of Targeting DRiP and Directing INS mRNA Breakdown

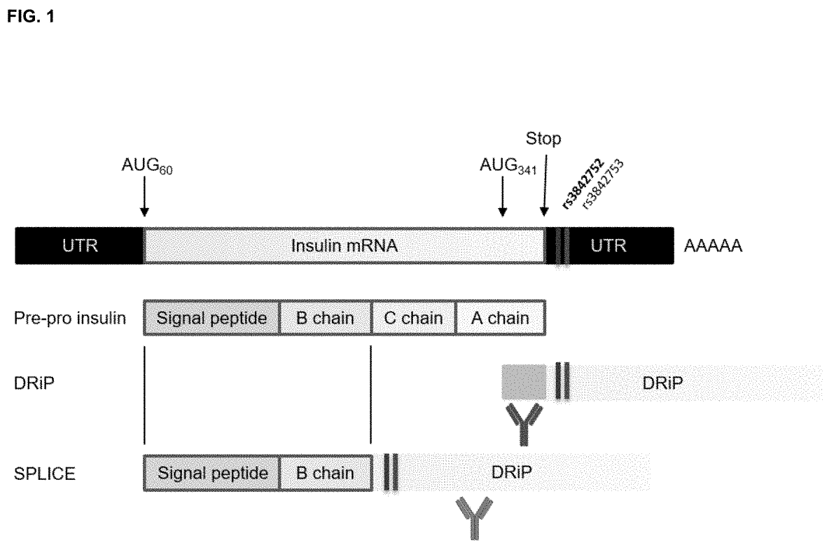

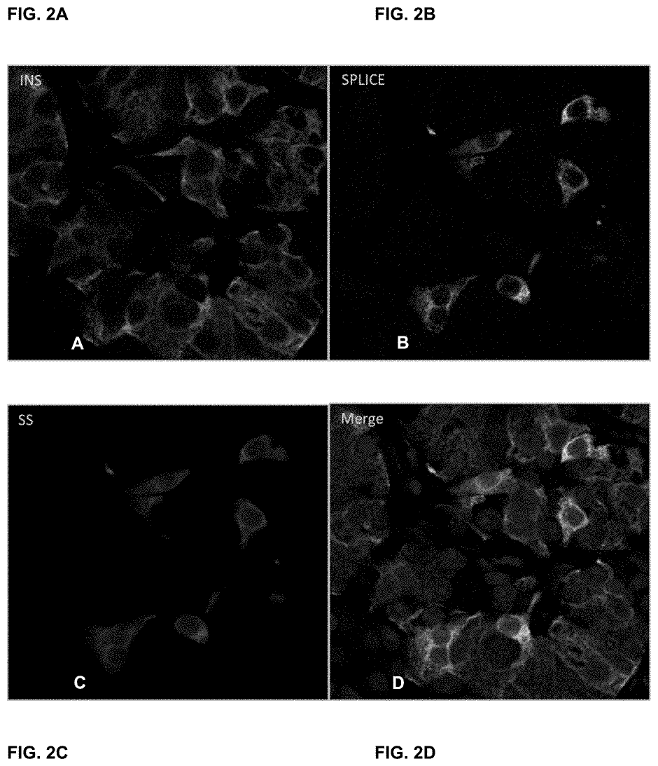

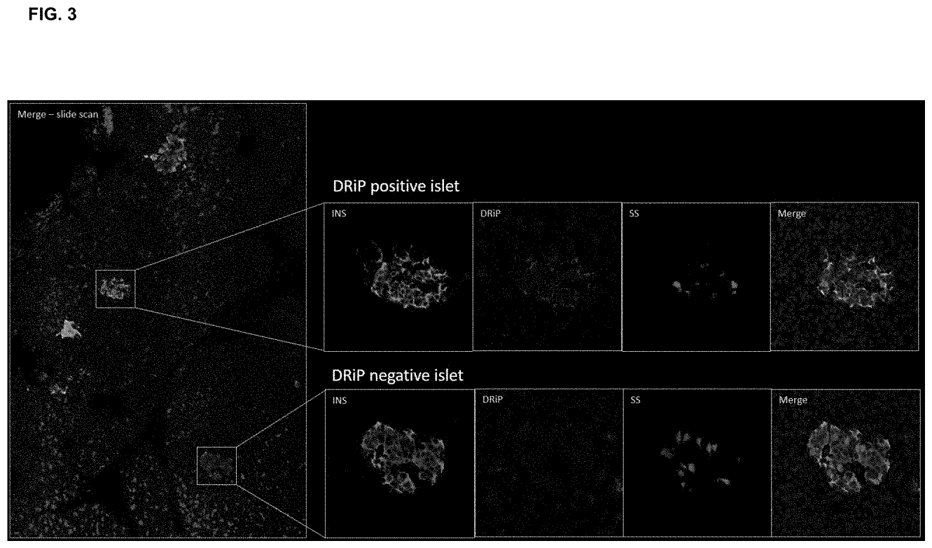

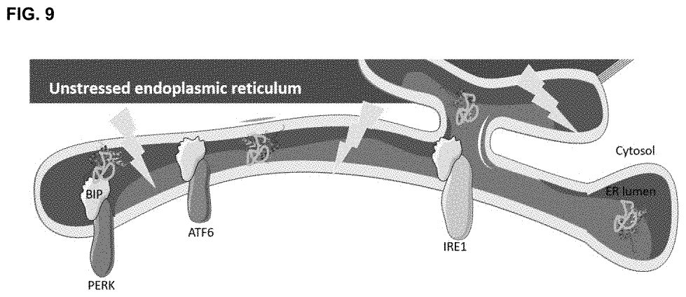

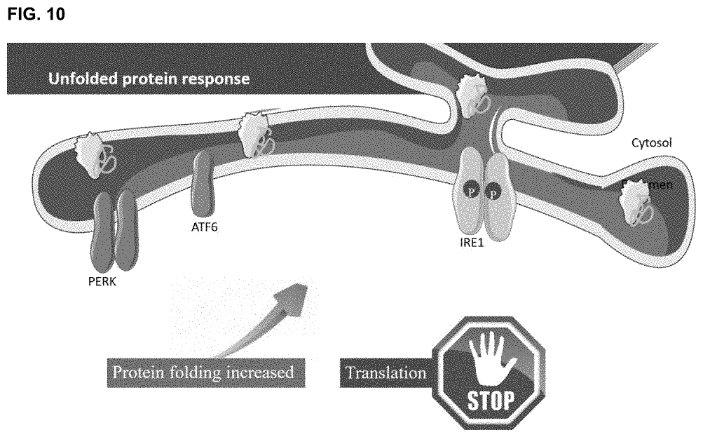

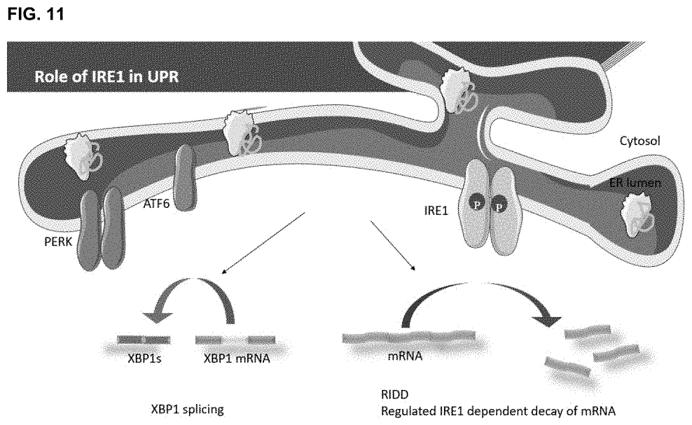

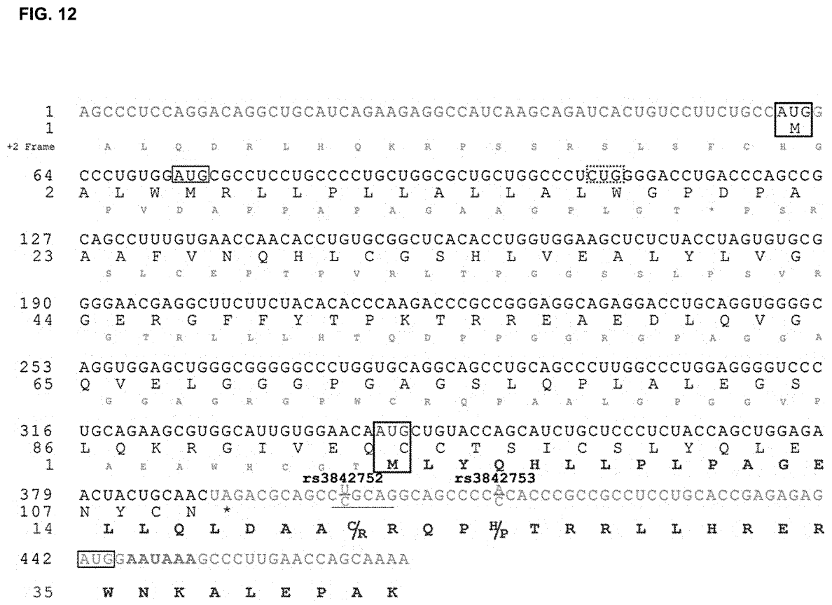

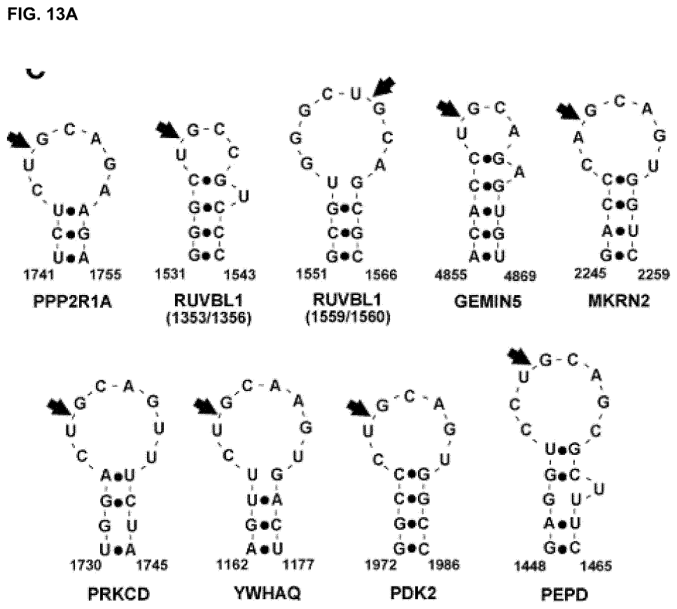

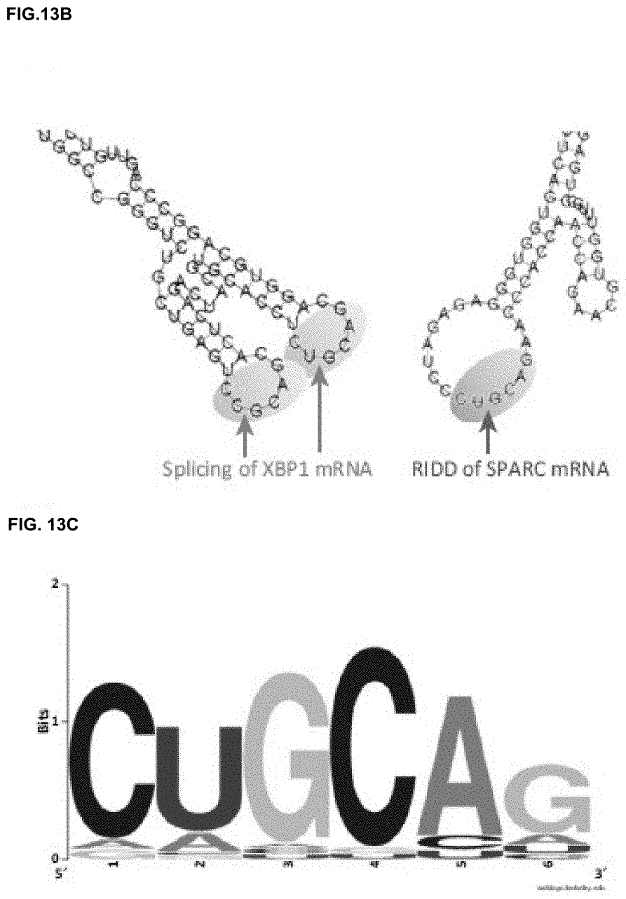

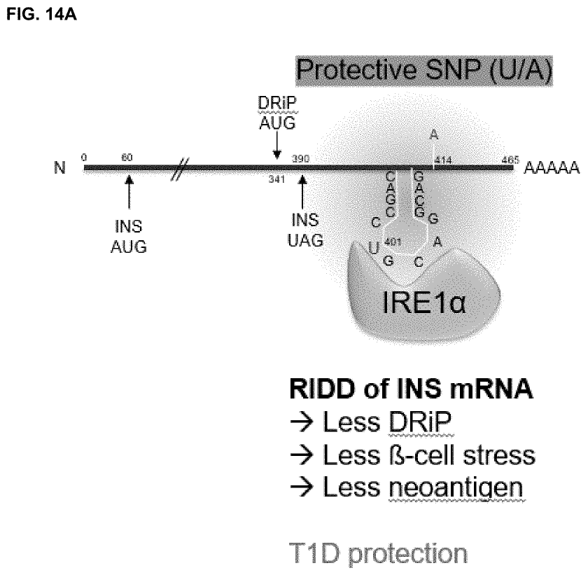

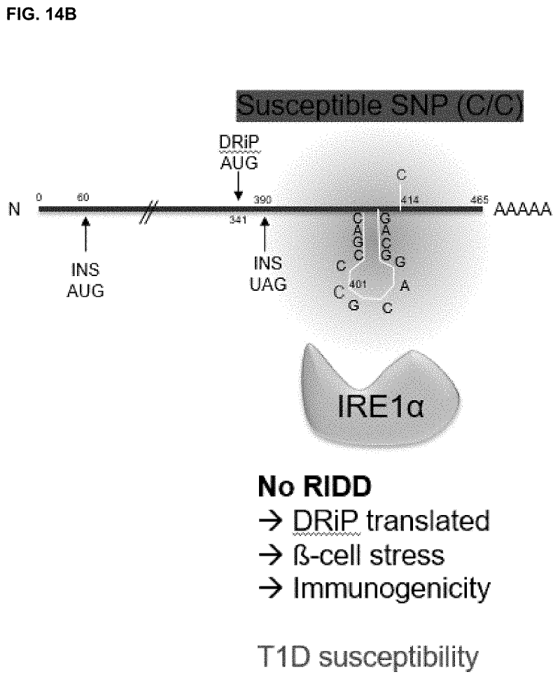

As disclosed herein, therapeutic targeting of INS mRNA breakdown may confer the benefits of genetic protein from T1D in individuals otherwise lacking protective INS SNPs. SNPs rs3842752 and rs3842753 are located within the 3′ UTR of the INS gene. See FIG. 1. As depicted in FIG. 12, SNP rs3842752 has two alternative sequences: CUGCAG (SEQ ID NO:1) or CCGCAG (SEQ ID NO:15). The protective SNP rs3842752 includes a U/A at position 401 whereas the susceptible SNP includes a C/C at position 401. See FIGS. 12, 14. During the unfolded protein response (UPR), a transmembrane protein named inositol-requiring enzyme 1 (IRE1) acts to splice XBP1 mRNA and carries out regulated IRE1 dependent decay of mRNA (RIDD). See FIGS. 9-11. In individuals with the rs3842752 protective SNP (U/A), IRE1 carries out RIDD of INS mRNA leading to less DRiP, less beta cell stress, and less neoantigen resulting in T1D protection. Conversely, in individuals with the rs3842752 susceptible SNP (C/C), IRE1 does not carry out RIDD of the INS mRNA. This results in translation of DRiP, beta cell stress, and immunogenicity leading to T1D susceptibility. See FIG. 14.

According to the embodiments herein, any suitable therapeutic targeting molecules may be used to target and reduce the production or activity of DRiP in a target cell (e.g., beta cell, islet cell, stem cell, donor cell) to reduce beta cell stress and to prevent or ameliorate T1D.

According to one embodiment, a gene editing technique may be used in a target cell to replace a susceptible sequence with a protective sequence at the rs3842752 site. In one example, the gene editing technique may involve the use of CRISPR/Cas, which involves using a donor template to replace a sequence within a target gene along with a guide RNA strand to direct the donor to the desired site, as known in the art. Thus, in certain embodiments, a targeting molecule for inhibiting DRiP expression is provided, which comprises a donor template that includes a nucleic acid sequence encoding an mRNA sequence CUGCAG (SEQ ID NO:1) to replace a single nucleotide polymorphism in the 3′ UTR of the INS gene. Any suitable gene editing technique may be used. In certain embodiments, CRISPR/Cas-mediated methods are used to deliver the donor template comprising a nucleic acid sequence that encodes an mRNA sequence CUGCAG (SEQ ID NO:1) to replace the rs3842752 susceptible (C/C) SNP in the 3′ UTR of the INS gene (e.g., replace the C residue at position 401 with a T residue). In those embodiments, the sequences may be DNA and may be single stranded or double stranded. Other technologies used in gene editing techniques that may be used according to the embodiments herein include transcription activator-like effector nucleases (TALENs), zinc-finger nucleases (ZFNs), and homing endonucleases or meganucleases.

In some embodiments, a gene editing technique may be used in a target cell to knock out the AUG341 thereby preventing DRiP expression. In other embodiments, a gene editing technique may be used to replace a susceptible sequence with a protective sequence at the rs3842752 and rs3842753 site. In other embodiments, a gene editing technique may be used to knock out or delete AUG341 and also replace a susceptible sequence with a protective sequence at the rs3842752 site.

According to other embodiments, molecules involved in RNA interference (RNAi) or antisense oligonucleotide technology may be used to block translation of DRiP. In such embodiments, an siRNA (or other RNAi molecule), an antisense oligonucleotide, or a cDNA molecule may be designed to hybridize to a portion of INS mRNA such that is blocks translation of DRiP. For example, in some embodiments, a nucleic acid sequence that is fully or partially complementary to at least a portion of INS mRNA is provided.

In certain embodiments, the nucleic acid sequence that is fully or partially complementary to at least a portion of INS mRNA is the complete INS mRNA. In certain embodiments, the nucleic acid sequence that is fully or partially complementary to at least a portion of INS mRNA comprises a nucleic acid sequence beginning at AUG341 in the INS gene. In certain embodiments, the nucleic acid sequence that is fully or partially complementary to at least a portion of INS mRNA comprises a nucleic acid sequence beginning at AUG₆₀ in the INS gene. In certain embodiments, the nucleic acid sequence that is fully or partially complementary to at least a portion of INS mRNA comprises a nucleic acid sequence beginning at AUG72 in the INS gene.

In other embodiments, a targeting molecule may be designed to target and bind DRiP to inhibit its activity. For example, in one embodiment, the targeting molecule may be an antibody or fragment thereof that binds a portion of DRiP (e.g., the immunogenic epitope of DRiP), thereby blocking recognition by the host immune system and preventing an autoimmune response. As described herein, an antibody fragment may be any suitable active fragment including, but not limited to, a single-chain variable fragment (scFv), a Fab fragment, a humanized antibody, an optimized antibody, or any suitable antibody fragment. In another aspect, the antibody or fragment thereof may be delivered to the cell in a way that it prevents expression of DRiP or prevents its antigenic presentation by the MHC. In other embodiments, the targeting molecule for inhibiting DRiP activity is a peptide or nucleic acid molecule that binds to DRiP to block an autoimmune response or prevent MHC presentation of a DRiP antigen. In certain embodiments, the nucleic acid that binds to DRiP is an aptamer.

The targeting molecules as described herein may be administered via any appropriate delivery mechanism. For example, the targeting molecules may be administered as naked DNA, or as part of a plasmid or vector. In some embodiments, a plasmid delivery may be used to deliver the targeting molecule wherein the plasmid is a silencer plasmid that is induced only when beta cells are in stress. Where nucleic acids are present in a vector, the vector may be viral or non-viral. DNA- and RNA-liposome complex formations are examples of useful non-viral vectors. Such complexes comprise a mixture of lipids which bind to genetic material (DNA or RNA), providing a hydrophobic coat which allows the genetic material to be delivered into cells. Liposomes which can be used include DOPE (dioleyl phosphatidyl ethanol amine) and CUDMEDA (N-(5-cholestrum-3-βol 3-urethanyl)-N′,N′dimethylethylene diam ine).

When the targeting molecule is administered using a liposome, it is preferable to first determine in vitro the optimal DNA: lipid ratio and the absolute concentration of DNA and lipid as a function of cell death and transformation efficiency for the particular type of cell to be transformed. These values can then be used, or extrapolated for use, in vivo administration. The in vitro determination of these values can be readily carried out using techniques known in the art. Some other examples of non-viral vectors include non-lipid cationic polymers [polyethylenimine (PEI), polyamidoamine (PAMAM), poly-Llysine], hemagglutinating virus of Japan-envelope (HVJ-E, an inactivated Sendai virus envelope), cationic liposomal lipid (Lipofectamine), and cationic non-liposomal lipids (Effectene), were developed for favorable transfection efficiency in gene transfer.

Other non-viral vectors can also be used in accordance with the present disclosure. These include chemical formulations of nucleic acids coupled to a carrier molecule or other molecule which facilitates delivery to target cells and tissues for the purpose of altering the biological properties of the host cells (e.g., increasing insulin secretion or sensitivity).

Exemplary protein carrier molecules include antibodies specific to the islet cells or receptor ligands, i.e., molecules and peptides capable of interacting with receptors associated with a cell of a targeted secretory gland.

Other methods for delivering nucleic acids to beta cells include, for example, lipoplex condensation and encapsulation, polymersome condensation and encapsulation, polyplex complex formation, dendrimer complex formation, inorganic nanoparticle complex formation, and cell penetrating peptide complex formation. These are further examples of possible chemical modifications of a nucleic acid described herein, facilitating delivery of the nucleic acid to a target cell or tissue. This delivery may be further facilitated by incorporating a beta cell specific peptide, thereby targeting the targeting molecule or pharmaceutical composition particularly to beta cells.

Nucleic acids describe herein can also be administered to a subject in a viral vector. The viral vector can be a lentiviral vector, such as a human immunodeficiency virus (HIV) vector or a simian immunodeficiency virus (SIV) vector, an adenoviral vector, or an AAV vector.

### Pharmaceutical Compositions

Also disclosed herein are pharmaceutical compositions comprising a targeting molecule as described herein and one or more suitable carriers or excipients. Examples of suitable carriers may include physiologically acceptable solutions, such as sterile saline and buffered saline. The excipient can be a natural or synthetic substance, and can act as a filler or diluents for the at least one nucleic acid, facilitating administration to the subject. The excipient can also facilitate nucleic acid uptake into a target cell, or otherwise enhance the effectiveness of the targeting molecule.

In some embodiments, the pharmaceutical composition may include a pharmaceutically effective amount of an adjuvant. Any immunologic adjuvant that may stimulate the immune system and increase the response to a vaccine or pharmaceutical composition, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Alum, Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosin al. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the pharmaceutical composition of the invention, as discussed above, may enhance the therapeutic effect achieved by the administration of the pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

In some embodiments, pharmaceutical composition described herein may be used in combination with other known pharmaceutical products, and may further comprise other drugs and additives. Examples of drugs or additives that may be used in conjunction with a pharmaceutical composition described herein include drugs that aid intracellular uptake of the composition disclosed herein, liposome and other drugs and/or additives that facilitate transfection, (e.g., fluorocarbon emulsifiers, cochleates, tubules, golden particles, biodegradable microspheres, and cationic polymers).

In some embodiments, the pharmaceutical composition described herein may be administered by directly injecting the therapeutic targeting molecules in PBS (phosphate buffered saline) or saline into a local site, by nasal or respiratory inhalation, or by intravascular (i.v.) (e.g., intra-arterial, intravenous, and portal venous), subcutaneous (s.c.), intracutaneous (i.c.), intradermal (i.d.), or intraperitoneal (i.p.) administration. The pharmaceutical composition of the present invention may be administered more than once.

### Methods of Use

The therapeutic targeting molecules described above may be used in methods for treating or preventing type 1 diabetes (T1D).

In some methods, treatment or prevention of T1D may include a step of administering a therapeutically effective amount of a targeting molecule or pharmaceutical composition thereof to a subject suspected of having a susceptible rs3842752 SNP mutation or lacking a protective rs3842752 SNP mutation.

In some embodiments, the therapeutic targeting molecules described above may also be used in methods for inhibiting the expression or activity of defective ribosomal products (DRiPs) in pancreatic beta cells. Those methods may be in vivo, in vitro, or ex vivo methods, and may include a step of contacting the pancreatic beta cells with an effective amount or therapeutically effective amount of the targeting molecule.

In some embodiments described herein, the therapeutic targeting molecules may be delivered using a plasmid or vector as described above. In some embodiments, a silencer plasmid may be administered to a healthy subject or beta cell that has a susceptible rs3842752 SNP mutation or lacks a protective rs3842752 SNP mutation such that the therapeutic targeting molecule (e.g., an siRNA molecule) is only induced with the beta cells are in stress, thereby preventing onset of T1D by blocking production of DRiPs during stress.

In other embodiments, methods for treating or preventing T1D may include transplantation of donor cells carrying the protective rs3842752 SNP (i.e., the protective INS variant) to a patient having T1D.

In certain embodiments, the donor cells may be genotyped prior to transplantation to select for cells that naturally carry the protective INS variant. In other embodiments, donor cells may be modified to (i) delete or knock-out the AUG341 in the INS gene to prevent expression of DRiP, (ii) replace a susceptible INS variant sequence with a protective INS variant sequence using a gene editing method as discussed above (e.g., using CRISPR/Cas, Zinc finger, or other gene editing methods known in the art), or both (i) and (ii). In some embodiments, the donor cells are contacted with an effective amount of a targeting molecule as discussed above in order to edit or inhibit the INS gene. The donor cells

According to some embodiments, the donor cells used in the methods described herein may be from a donor pancreas (e.g., donor islets or beta cells) or they may be beta cells derived from (i.e., differentiated from) donor stem cells (e.g., induced pluripotent stem cells, embryonic stem cells). According to certain embodiments, the donor cells may be used in an autologous transplant (i.e., the donor cells are obtained from the same individual receiving the transplant), an allogenic transplant (i.e., the donor cells are from a different individual than the individual receiving the transplant), or a heterologous transplant (i.e., the donor cells are from a mixed population of donor cells).

As discussed above, the donor cells used in the methods discussed above should carry the protective INS variant. That is because it was determined that insulin gene (INS) variation and beta cell stress associate with risk for development of type 1 diabetes (T1D). To alleviate ER stress, the unfolded protein response is initiated involving activation of inositol-requiring enzyme 1 (IRE1α), which impedes translation by mRNA decay. Further, it was determined that the protective insulin mRNA variant harbors the motif required for IRE1α, leading to accelerated insulin mRNA decay during ER stress compared to the susceptible insulin variant. Islets of human donors with protective INS variant show improved vitality and function, and reverse diabetes more rapidly when transplanted into diabetic mice than islets carrying susceptible INS variants only. As discussed in the working examples below, an explanation for genetic protection from T1 D inferred by INS variation involving peripheral regulation was identified, which may act in concert with a previously proposed mechanism of central tolerance, and reducing translational burden, neoantigen formation and immunogenicity. Consequently, only donor cells with the protective INS variant should be used as donors in beta cell replacement therapy in T1D.

In other embodiments, the methods for treating or preventing T1D include an in vivo targeting method, wherein a targeting molecule is delivered to target cells using liposomes, aptamers, or other techniques known for targeting and delivering therapeutics to beta cells in vivo.

The term “an effective amount” as used herein refers to an amount of a composition that produces a desired effect. For example, a population of cells may be contacted with an effective amount of the therapeutic targeting molecules described herein to study its effect in vitro (e.g., cell culture) or to produce a desired therapeutic effect ex vivo or in vitro. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the com position (including activity, pharm acokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the composition is administered alone or in combination with another composition, drug, therapy or other therapeutic method or modality. One skilled in the clinical and pharmacological arts will be able to determine an effective amount or therapeutically effective amount through routine experimentation, namely by monitoring a cell's or subject's response to administration of a composition and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, Pa., 2005, which is hereby incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

### INS Variants and Insulinoma

While T1D is a disease that results in insufficient insulin production, insulinoma is a cancer that results in too much insulin production. This reflects a situation where in autoimmune disease the immune response is too strong, leading to killing of beta cells by autoreactive T cells, whereas during insulinoma the immune response is too weak giving cancerous beta cells a change to proliferate out of control. As discussed below, this may also reflect inverse genetic relationships between type 1 diabetes (T1D) and cancer, including an inverse relationship with respect to the susceptible and protective INS variants. In particular, whereas the neoantigens produced by the susceptible INS variant may cause an increased autoimmune activity that is detrimental to normal beta cells, those neoantigens may present a target for identifying and targeting tumor cells.

Thus, in some embodiments, methods for identifying, targeting, treating, and diagnosing insulinomas and other cancers are provided. Such methods may include contacting a population of cells suspected of being cancerous (e.g., islet or beta cells) with a target molecule (e.g., an antibody of oligonucleotide) that binds to a neoantigen produced by a susceptible INS variant. Upon identifying the neoantigen, treatment using an antibody against DRiP may be administered to a subject producing the neoantigen. Alternatively, the neoantigen may be used in a vaccine or immunogenic composition to induce an immune response to and/or generate antibodies against cells expressing or presenting the neoantigen.

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLES

### Example 1

### Autoimmunity Shown Against a Defective Ribosomal Insulin Gene Product in Type 1 Diabetes

Human pancreatic beta cells are insulin factories dedicated to the maintenance of glucose homeostasis; insulin, stored in secretory granules, represents 10-15% of the protein content of these cells. Upon glucose challenge, insulin molecules are released into the circulation by exocytosis, and insulin mRNA is rapidly translated by polysomes to increase insulin biosynthesis. Studies of samples from humans with T1D and mouse models of the disease indicate that native insulin and its precursors act as primary autoantigens, and fragments of the signal peptide of the preproinsulin (PPI) were identified as main targets of cytotoxic islet-autoreactive CD8⁺ T cells in human T1D27. In a process similar to the emergence of DRiPs during tumor development, the high demand for insulin may lead to the generation of aberrant insulin polypeptides by impaired translation fidelity, rendering beta cells immunogenic. These errors may yield antigens that trigger or accelerate islet autoimmunity in T1D, particularly as a result of stresses that, for example, may be evoked by viral infection, reduced pancreas size or inflammation. Here, it was investigated whether beta cells produce aberrant translation products that can lead to the generation of diabetogenic epitopes.

Leaky ribosome scanning for translation initiation at a downstream AUG can generate out-of-frame translation products. Within the human insulin mRNA, two putative downstream translation initiation sites with strong Kozak consensus sequences are located at positions 72 and 341. Although translation initiation on the AUG at position 72 (which is in frame with the canonical AUG of PPI) would produce a truncated isoform of PPI, initiation on the AUG at position 341 (AUG341) would generate an alternative polypeptide in a +2 reading frame that does not share any sequence identity with the canonical translation product of the insulin gene. The absence of a stop codon in this frame would lead to translation into the poly(A) tail. It was shown that initiation can occur at AUG341 by generating green fluorescent protein (GFP) fusion constructs in which the GFP coding sequence was fused in frame with the bona fide PPI AUG₆₀ (insulin (INS)-GFP) or with the alternative AUG at position 341 (INS-DRiP-GFP). As a control, a third construct was generated, in which the alternative reading frame was cloned immediately after the CMV promoter (DRiP-GFP). Following transfection in HEK 293T cells, in order to achieve high protein expression, both INS-GFP and INS-DRiP-GFP constructs led to expression of GFP fusion proteins migrating at 40 kDa and 33 kDa, respectively. The control construct, DRiP-GFP, confirmed the molecular weight of the unconventional product. Western blots and immunohistochemistry confirmed that cells transfected with INS-DRiP-GFP also coexpress proinsulin.

The effect of endoplasmic reticulum stress (ER stress) on translation initiation was also determined. Cells transfected with INS-DRiP-GFP were stimulated with the ER stress-inducers thapsigargin (TG) or tunicamycin (TM). The expression of DRiP polypeptide was increased by TG but not TM treatment as quantified both by western blot and flow cytometry analysis. Altogether, these data indicated that the production of the INS-DRiP polypeptide may result from calcium depletion in the ER and the subsequent increase in levels of cytoplasmic calcium, rather than the classical ER stress caused by accumulation of misfolded proteins as induced by N-linked glycosylation inhibition (TM).

On the basis of the insulin sequence, translation of this neopolypeptide continues beyond the original PPI stop codon and leads to translation of two single nucleotide polymorphisms (SNPs) (rs3842752 and rs3842753) that are in strong linkage disequilibrium and are associated with development of T1D. Although these SNPs have been judged to be functionally irrelevant because of their location within the INS 3′ UTR, in this scenario, the SNPs rs3842752 and rs3842753 may generate four different polypeptide variants of the INS-DRiP polypeptide containing, respectively, cysteine-to-arginine (C-to-R) and histidine-to-proline (H-to-P) substitutions that may act as neoantigens in T1D. To test the immunogenicity of the INS-DRiP variants, T cell proliferation assays were performed on freshly isolated peripheral blood mononuclear cells (PBMCs) of juveniles with T1D for detection of islet autoimmunity. Proliferative responses were detected in the majority of T1D cases that strongly correlated in reactivity between the ‘susceptible’ (R-P) and ‘protective’ (C-H) INS-DRiP variant, suggesting that the SNP region itself did not contribute to immunogenicity and underscoring the lack of presentation of the SNP by HLA-DQ. Intriguingly, the few individuals carrying genetic risk variants associated with protection from T1D (HLA-DQ6.2 or INS SNP C-H) did not respond to INS-DRiP polypeptides, supporting the hypothesis that DQ6.2 and INS SNP C-H contribute to central immune tolerance in thymic education. Strong T cell responses to INS-DRiP were detected in individuals with increased genetic risk for T1D (those heterozygous for HLA-DQ2 and HLA-DQ8 (HLA-DQ2/8); i.e., HLA-DQ8trans (formed by the α-chain of HLA-DQ2 (DQA1*05:01) and the β-chain of HLA-DQ8 (DQB1*03:02)) or HLA-DQ2trans (formed by the α-chain of HLA-DQ8 (DQA1*03:01) and the β-chain of HLA-DQ2 (DQB1*02:01)). In these studies, it was observed that the T cell response to INS-DRIP was similar in amplitude in comparison to the other known antigen targets in T1D.

Previous studies examined whether dendritic cells (DC), which are uniquely capable of priming the immune system, were able to process and present peptides from the INS-DRiP polypeptide. Monocyte-derived, immature DC generated from DQ2- or DQ8-homozygous or DQ2/8-heterozygous donors were pulsed with the INS-DRiP variants. After 24 h of culture, the DC HLA-DQ ligandome was determined by mass spectrometry, revealing a short peptide fragment of 9 amino acids from the N-terminus of the INS-DRiP polypeptide as a single candidate epitope. A full overlap of the experimental fragmentation profile of the identified peptide and its synthetic counterpart confirmed proper MS identification. Cell-free HLA-DQ binding studies confirmed a strong binding affinity of the 9-mer peptide to HLA- DQ8cis and HLA-DQ8trans molecules but not to HLA-DQ2cis or HLA-DQ2trans molecules. These data show that the INS-DRiP peptide fragment is naturally processed by DCs and is preferentially presented by T1D highest-risk HLA-DQ8trans, which is expressed in DQ2/8 heterozygous individuals. Also, monitoring the immune response to INS-DRiP₁₋₉ by interferon (IFN)-γ and GrzB Elispot indicates that the peptide-stimulated production of granzyme B occurs exclusively in individuals with T1D. Together, these results indicate that the N-terminus of the INS-DRiP1-9 polypeptide is particularly immunogenic and suggests that a cytotoxic T lymphocyte (CTL) response occurs in T1D.

Previous studies screened the INS-DRiP polypeptide for potential human leukocyte antigen (HLA)-class-1-binding epitopes. The INS-DRiP sequence was analyzed by three major histocompatibility complex (MHC) class I epitope prediction algorithms: NetMHC 3.4, SYFPEITHI and BIMAS. One strong HLA-A2-binding peptide was identified that was identical to the INS-DRiP₁₋₉ eluted from HLA-DQ8trans. The INS-DRiP₁₋₉ epitope MLYQHLLPL (SEQ ID NO:2) was confirmed to bind with high affinity to HLA-A2, which is the most prevalent HLA variant within the T1D population.

To assess the clinical relevance of the INS-DRiP₁₋₉ epitope, previous studies explored the presence of specific CD8⁺ T cells in peripheral blood samples from HLA-A2⁺ individuals with T1 D and in HLA- and age-matched, healthy donors. Significantly higher levels of INS-DRiP₁₋₉-specific CD8⁺ T cells were detected in individuals with T1D as compared to healthy donors, whereas antiviral CD8⁺ T cell frequencies were similar between the two groups. Phenotypical analysis of INS-DRiP₁₋₉-Tm⁺ CD8⁺ T cells showed more cells with the effector phenotype (CCR7⁻CD45RA⁻) in donors with T1D than in healthy donors.

These T cells were further characterized by generating INS-DRiP₁₋₉-specific T cell clones from PBMCs of individuals with T1D by dual HLA tetramer staining, and their cytotoxic properties were determined on peptide-loaded HLA-A2⁺ JY cells. These DRiP-specific CTL clones were generated from an individual with long-term diabetes (clone #1) as well as an individual with new-onset diabetes (clone #2) indicating that such T cells are participating at different stages of disease progression. Complete lysis of peptide-pulsed target cells was detected, whereas target cells loaded with an irrelevant peptide remained unaffected. Peptide-specific T cell activation resulted in a significant increase in the secretion of IFN-γ and tumor necrosis factor (TNF)-α—and, to a lesser extent, of macrophage inflammatory protein (MIP)-1β—when compared to unstimulated T cells. Next, cytotoxic potential of the INS-DRiP₁₋₉-specific CTL on human islet cells expressing HLA-A2 was examined. In order to specifically investigate beta cell death, dispersed primary human islets were transduced by a lentivirus containing the beta-cell-specific viability reporter (i.e., a short-half-life luciferase reporter gene under the control of the human insulin promoter, HIP-LUC2CP) as previously described. Following LV-HIP-LUC2CP transduction, human pancreatic islet cells were incubated with CTLs specific for INS-DRiP₁₋₉, CMVpp65 or PPI15-24, and cytolysis was determined by measuring reduction of luciferase activity. PPI15-24-specific and INS-DRiP₁₋₉-specific CTLs significantly reduced beta cell survival, whereas beta cell survival was unaffected by CMV-specific CTLs. Together, beta cell destruction by the INS-DRiP₁₋₉-directed CTLs confirmed that the DRiP₁₋₉ epitope is naturally generated, processed and presented on the cellular surface of human beta cells. The partial destruction of beta cells by INS-DRiP₁₋₉-specific CTLs is in tune with the error hypothesis and points to beta cell heterogeneity and immunogenicity; this is perhaps due to differential sensitivity to stress factors. To test whether inflammation increases beta cell susceptibility to cytolysis by INS-DRiP₁₋₉-specific T cells, beta cells were preconditioned with a medium containing high glucose in combination with the proinflammatory cytokines IL-1β and IFN-γ to mimic T1D pathology. As anticipated from the results regarding the effect of ER stress on DRiP translation described above, these inflammatory conditions further increased beta cell death in the presence of INS-DRiP₁₋₉-specific CTLs. INS-DRiP₁₋₉-specific CTL cocultured with human islets consistently secreted the effector cytokines MIP-1β, IFN-γ and TNF when recognizing the INS-DRiP epitope presented by HLA-A2, corroborating specific stimulation and activation of the CD8⁺ T cells by islets cells.

The studies described above (1) present the first evidence of a naturally processed and presented epitope derived from nonconventional islet proteins leading to the destruction of human beta cells by cytotoxic CD8⁺ T cells; (2) identified an immunogenic polypeptide translated from the insulin mRNA that is capable of triggering T cell proliferation in PBMCs from individuals with the highest odds ratio for T1D; and (3) demonstrated that the epitope, presented by HLA-A2 as well as the highest-T1D-risk HLA-DQ8trans molecules expressed on DQ2/8 heterozygous DCs, is implicated in T1D pathogenesis as patient-derived CTLs specific for this epitope are able to kill human beta cells in vitro. The presence of phenotypically naive DRiP₁₋₉-specific CTLs in healthy individuals and memory T cells in individuals with T1D indicates that neoantigen-specific CTLs are part of the normal T cell repertoire, and that the higher frequency and activated phenotype of these CTLs detected in PBMCs of individuals with T1D points to peripheral activation linking DRiP₁₋₉-specific CTLs with the immunopathogenesis of T1D.

The studies discussed above propose a new pathway of beta cell destruction by the immune system in which the generation of the neoepitope, such as INS-DRiP₁₋₉, plays a central role. The mechanisms regulating the synthesis of the INS-DRiP polypeptide are still unknown, but two scenarios can be envisaged. In the first model, exon 2 of the insulin gene would be spliced from the pre-mRNA, thus positioning the AUG341 as the first AUG encountered by the ribosome during scanning. Alternatively, the ribosome scan of the canonical AUG may result in translation initiation at a downstream AUG in the mature insulin mRNA. Such splicing variants in mRNA analysis of human pancreatic islets under either normal or pathogenic conditions were not detected in previous experiments. This indicates that the alternative translation initiation is the most plausible mechanism. Environmental modifications leading to ER stress appear to be an important component that can control expression of an alternative reading frame as described for other stress-induced proteins (i.e., ATF4 and ATF5). The fact that human beta cells are exceptionally sensitive to ER stress reinforces this finding. In addition to an effect on translation initiation processes, environmental stress may also have an impact on the degradation of insulin byproducts. The absence of an in-frame stop codon in the INS-DRiP protein is peculiar and may imply the participation of distinct elimination mechanisms that are engaged in clearing nonstop proteins. The implication of translational mistakes in autoimmunity shown here begs for revision of transcriptome-based approaches for epitope discovery and offers alternatives for tissue- and antigen-specific therapeutic approaches aiming at the induction of immune tolerance. The findings described above further support the emerging concept that beta cells are destroyed in T1D by a mechanism comparable to classical antitumor responses whereby the immune system has been trained to survey for dysfunctional cells in which errors have accum ulated.

### Example 2

### Donor SNP Status May Correlate with Type-1-Diabetes Diagnosis

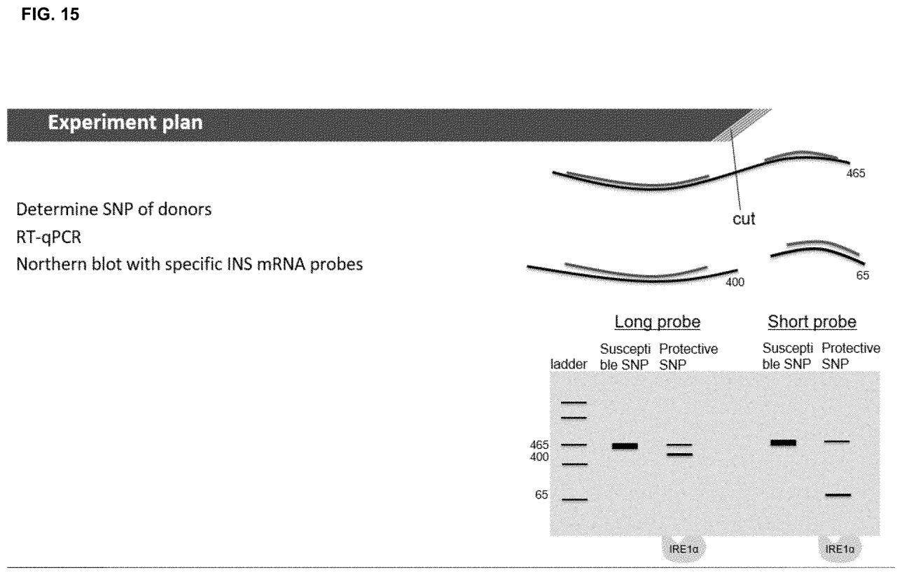

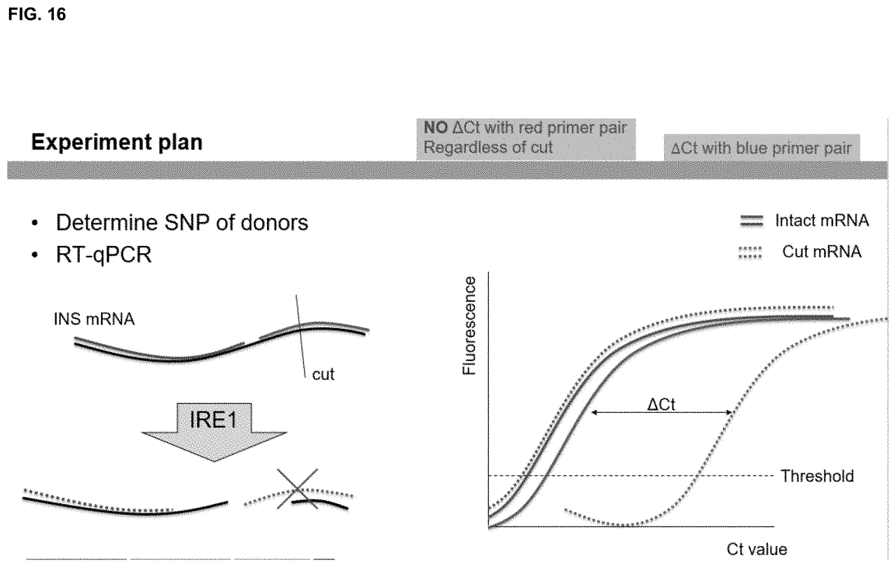

Donors were tested to determine whether a protective or susceptible SNP (rs3842752) was present. To do so, a sample was obtained from the donors. mRNAwas extracted from each sample and treated with IRE1α to determine whether the subject's INS mRNA is cut or intact (see FIG. 15). Additional experiments may be run to confirm a subject's SNP status (cut or intact mRNA) (see FIG. 16).

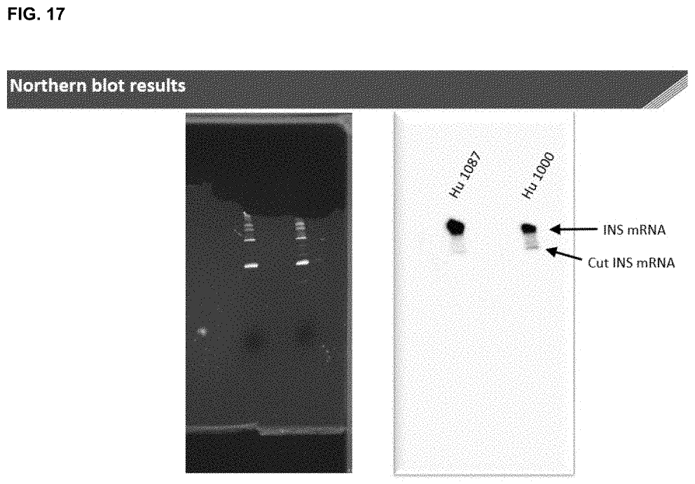

As shown in FIG. 17, subject Hu 1087 showed an uncut INS mRNA, indicating a susceptible SNP, while subject Hu 1000 showed a cut INS mRNA, indicating a protective SNP. Based on these results, one may predict that Hu 1087 has T1D or is at risk for developing T1D and is a candidate for therapeutic interventions described above.

### Example 3

### IRE1-Mediated Cleavage of Insulin mRNA Relieves Beta Cell Stress in Protective SNP Variant

The following studies were performed to determine whether IRE1 cleaves protective SNP insulin mRNA (U/A) more efficiently than the susceptible variant (C/C) (see FIG. 14), thereby relieving beta cell stress and protecting against T1D.

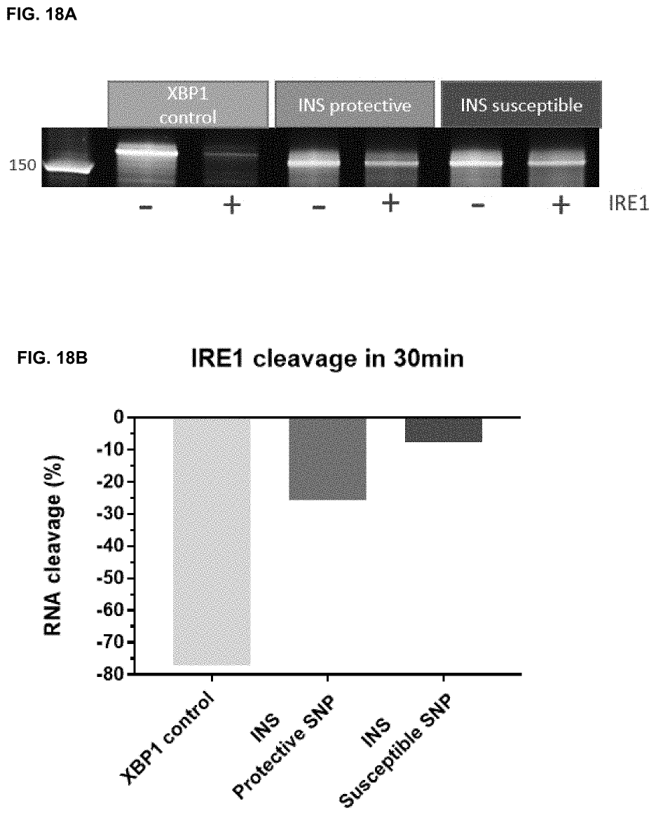

In vitro cleavage of insulin mRNA (in vitro produced) with both SNPs. First, cleavage of in vitro transcribed RNA by IRE1 was performed for both SNPs. A 166nt RNA strand-resembling XBP1 (control), INS^{protective} and INS^{susceptible}—were made by in vitro transcription and then incubated with or without IRE1 (see FIG. 18A). The results of the RNA cleavage products were then quantified. As shown in FIGS. 18A and 18B, in vitro cleavage by IRE1 was successful as seen by cleavage of XBP1 (IRE1's normal target). The protective variant of insulin RNA is cleaved more efficiently (25% cleaved in 30 minutes) compared to the susceptible insulin variant (7% cleaved in 30 minutes).

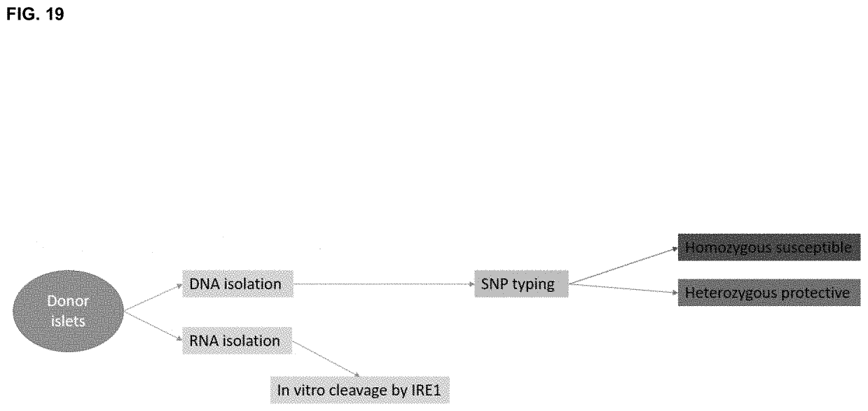

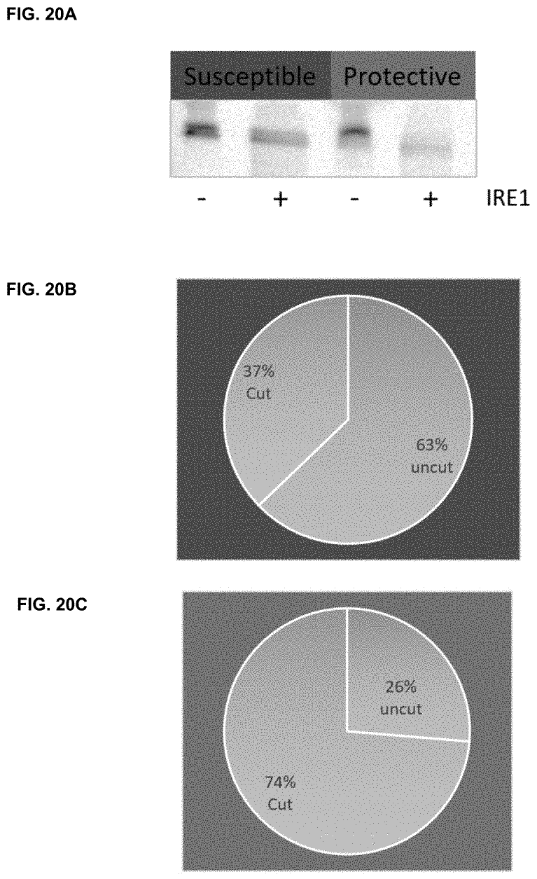

In vitro cleavage of insulin mRNA isolated from human islet donors. DNA and RNA was isolated from human donor islets for further processing (FIG. 19). The DNA was used to perform SNP typing, and each donor was determined to be homozygous susceptible or heterozygous protective. Total RNA for each donor was then incubated with or without IRE1 and resolved on a 10% gel. RNA was blotted to a membrane and probed with specific insulin probes (see FIG. 20A). Band intensity is shown in pie diagrams for susceptible (FIG. 20B) and protective (FIG. 20C) donors. As shown in FIGS, human insulin mRNA of the protective SNP donor is cut more efficiently in vitro than the susceptible variant.

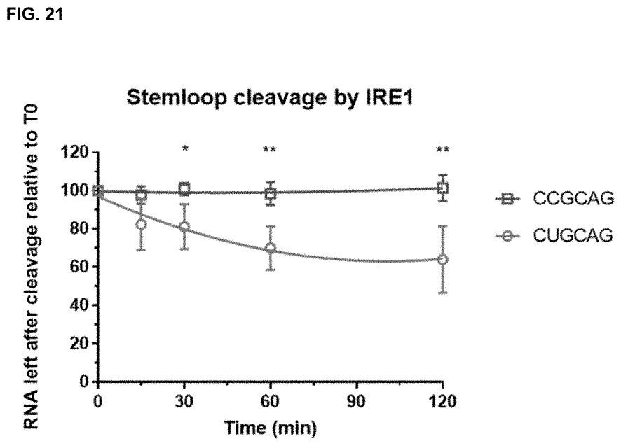

In vitro cleavage of RNA stemloop with both SNP variants. An 18nt piece of RNA stem loop of both the susceptible and protective insulin variant were cleaved in vitro with IRE1. As shown in FIG. 21, IRE1 cleaves the protective stem loop and not the susceptible stem loop.

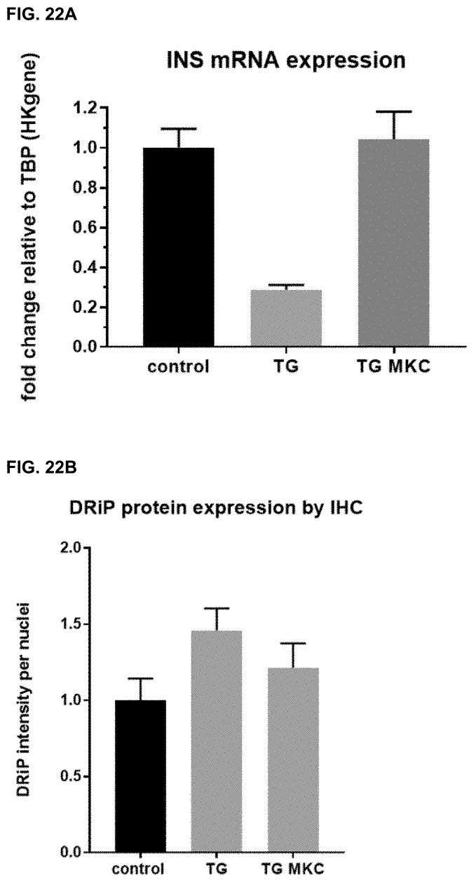

Ex vivo insulin mRNA expression and DRiP protein expression in beta cell line under stress. Ex vivo cleavage of insulin mRNA was examined in a 1.1B4 cell line. A human beta cell line (1.1B4) was stressed with Thapsigargin (chemical), with or without inhibition of IRE1, followed by analysis of the insulin mRNA expression and DRiP protein expression. As shown in FIG. 22A, Insulin RNA expression goes down when cells are stressed with TG. This phenomenon is reversed back to normal when IRE1 is inhibited with MKC, indicating that IRE1 gets activated by the stress and cleaves insulin mRNA. As shown in FIG. 22B, DRiP protein expression goes up when cells are stressed with TG, despite the decrease in insulin mRNA. These results indicate that IRE1 dependent decay of insulin mRNA takes place ex vivo, even in the susceptible SNP. But it is not enough to prevent an increase in DRiP expression. The protective SNP may have this effect ex vivo.

Discussion. The studies above indicate that IRE1 cleaves the protective SNP RNA (stem loop, RNA strand and human donor RNA) more efficiently then the susceptible SNP RNA according to in vitro cleavage data. Further, it was shown that IRE1 is the enzyme responsible for insulin mRNA cleavage ex vivo. Additional data and discussion may be found in the poster appended as Appendix A, attached hereto and incorporated by reference in its entirety. Enlarged panels corresponding to those shown in Appendix A are appended as Appendix B, which is attached hereto and incorporated by reference in its entirety.

With respect to therapeutics, there are no IRE1-related therapeutics currently available for the treatment of T1D. In Type 2 Diabetes (T2D) the role of the SNP is reversed, and the T1D protective SNP' makes individuals susceptible to T2D. Thus, in that case IRE1 may be blocked from cleaving the protective insulin mRNA, which might lead to the production of more insulin (low insulin levels are a problem in T2D). Blockage of IRE1 should be designed to specifically target beta cells since IRE1 is heterogeneously expressed in the body.

### Example 4

### The INS Variant Protecting from Type 1 Diabetes Associates with Accelerated Insulin mRNA decay, Reduced Immunogenicity and Improved Islet Function and Vitality

Type 1 diabetes (T1D) is a multifactorial autoimmune disease with a strong genetic component, leading to destruction of insulin producing pancreatic beta cells and loss of glycemic control. Next to HLA genes, genetic risk for T1D is strongly associated with insulin (INS) gene variation [Davies-Todd, 1994], including a variable number of tandem repeats (VNTR) and single nucleotide polymorphisms (SNP) that are in tight linkage disequilibrium [Bennett-Todd, 1995]. Increased thym is expression and decreased pancreatic expression of proinsulin mediated by the dominant protective haplotype (class III VNTR) has been claimed to explain central immune tolerance to proinsulin and protection from T1D [Polychronakos 1997; Pugliese, 1997; Durinovic-bello, 2005; Bennet, 1994; Vafiadis, 1996]. Beta cells contribute to their demise by upregulation of HLA class I, production of chemokines and ER stress. The generation of a stress-induced alternative INS product due to ribosomal infidelity that act as a strong neoantigen was recently described. This defective ribosomal INS product (INS-DRiP) results from alternative initiation of translation of INS mRNA in the +2 reading framing, leading to skipping of the canonical stop codon and resulting in translation of two SNPs (r53842752 and rs3842753) in the INS 3′ UTR into polymorphic proteins. CD8 T cells isolated from T1D patients that are reactive with INS-DRiP selectively destroy stressed beta cells. The rare cases developing T1D in spite of carrying a protective INS variant never responded to INS-DRiP. Since central tolerance against a neoantigen expressed in stressed islets but not thymus is unlikely, autoimmunity to INS-DRiP in individuals was tested, where protective INS variants are controlled by peripheral immune tolerance, acting in concert with central immune tolerance to proinsulin itself.

To alleviate ER stress and the accompanying INS-DRiP production, beta cells initiate the unfolded protein response (UPR) involving activation of inositol-requiring enzyme 1 (IRE1α). ER stress activates IRE1α by promoting dimerization, and autophosphorylation, resulting in increased ER chaperone expression, degradation of misfolded proteins and reduced translational burden by regulated IRE1α dependent decay (RIDD) of ER-localized mRNA. The RIDD process requires a specific mRNA motif (CUGCAG, SEQ ID NO:1) and hairpin structure. It was tested to determine whether human insulin mRNA variants could be substrate for IRE1α leading to insulin mRNA decay, decreased ER stress, neoantigen formation and immunogenicity, and improved beta cell function and vitality.

Methods

Cloning and dual luciferase reporter assay. The INS 3′ UTR (bp375-466) was cloned into pSICheck 2.2 vector using NEBuilder HiFi DNA assembly cloning kit (New England Biolabs) after digestion with Xhol (New England Biolabs). Clones were transduced into competent cells using NEBuilder HiFi DNA assembly chemical transformation (New England Biolabs). Next, vectors were isolated using zymopure II plasmid midiprep kit (Zymo Research), and sequenced to validate cloning and absence of mutations. HEK293T cells were cultured in RPMI and transfected with either vector using Lipofectamine 3000 (Thermo Fisher Scientific). After TG treatment (Abcam), the dual-luciferase reporter assay was performed according to protocol (Prom ega).

Human islets. Human islets were isolated as described previously [Ricordi et al., Diabetes, 31 Dec 1988, 38 Suppl 1:140-142 (DOI: 10.2337/diab.38.1.s140, PM ID: 2642838); Meirigeng et al., Transplant Direct. 2015 May;1(4):e14. doi: 10.1097/TXD.0000000000000522, PM ID: 26146662; Meirigeng et al., Transplant Direct. 2016 Feb;2(2):e63. doi: 10.1097/TXD.0000000000000574, PM ID: 26894230; Meirigeng et al., Am J Physiol Endocrinol Metab. 2015 Mar. 1; 308(5):E362-9. doi: 10.1152/ajpendo.00443.2014, PMID: 25537495] and cultured in Connaught Medical Research Laboratories (CMRL)-1066 media supplemented with 0.5% human serum albumin (Baxter Healthcare Corporation) and 0.1 μg/ml insulin-like growth factor-1 (Cell Sciences). Donors were genotyped for rs3842752 and rs3842753 by Sanger sequencing using INS-specific primers (Fw: 5′-GGAGGGGCTCACAACAGTG, SEQ ID NO:20 and Rv: 5′-GTGTCTCCCTGACTGTGTCC, SEQ ID NO:21). Eighteen islet preparations were used for DNA/RNA isolation using allprep DNA/RNA mini kit (Qiagen) and ex vivo and in vivo function analysis.

Islet preparations of 4 different donors (2 carrying protective INS variant and 2 with susceptible INS variant only) and/or ECN90 cells are cultured and treated with 2 mM TG for 8 h or inflammatory conditions for 24 h, after treatment cells were harvested. Harvested cells are then analyzed using immunohistochemistry (IHC), Western blot (WB) and ex vivo INS mRNA expression (RNA) during stress to assess metabolic stress, inflammatory stress, ER stress, and immunogenicity using the following markers:

| Method | Impact of Stress Markers | Immunogenicity Markers |
|---|---|---|
| RNA | INS cleavage | |
| WB | CHOP, XBP1s, BiP | B2 microglobulin, HLA-ABC |
| IHC | INS/proINS | DRiP |

In vitro cleavage assay and RT-qPCR. Whole islet mRNA (30 ng/ul) was incubated with recombinant 0.2 μg IRE1α (SignalChem Biotech Inc.) in cleavage buffer (20 mM HEPES, 50 mM NaCl, 1 mM DDT, 1 mM ATP) at 37° C. Samples were taken at the indicated time points followed by heat inactivation (3 min at 70° C.). cDNA was made using qscript cDNA SuperMix (QuantaBio) and Taqman qPCR was done using custom insulin specific primers and probe (Fw: 5′-CTGGAGAACTACTGCAACTAGAC, SEQ ID NO:22; Rv: 5′-TGCTGGTTCAAGGGCTTTAT, SEQ ID NO:23; and probe: 5′-CTCCTGCACCGAGAGAGATGGA, SEQ ID NO:24) (Applied Biosystems).

Ex vivo islet function. Islet oxygen consumption rate (OCR) was measured using a Seahorse XFe analyzer (Seahorse Bioscience). Briefly, islets were washed with modified Seahorse XFe assay media containing 3 mM glucose and 1% FBS, and equilibrated in same assay media for 3 hours at 37° C. Next, 70-100 IEQ islets were handpicked and plated into Seahorse XFe islet capture plates (Seahorse Bioscience). Islet OCR was measured at basal level (3 mM glucose), upon glucose stimulation (20 mM glucose), and on mitochondrial respiration inhibition (Oligomycin 5 nM). OCR fold increase was calculated by dividing OCR upon glucose stimulation by OCR at basal level. Minimum of 4 islet samples were measured simultaneously in each experiment.

For insulin secretion assay, 2-3 islet aliquots containing approximately 150 IEQ were used from each isolation. Insulin secretion was determined in low (3mM) and high (20mM) glucose by in vitro perifusion system following the standard operation procedure used by City of Hope [ref]. Insulin secretion of each effluent was measured using an ELISA kit for human insulin (ALPCO). Stimulation index (SI) was calculated by dividing the total insulin secreted in high glucose by the total insulin released in basal low glucose for the same period.

In vivo islet function. Transplantation of isolated human islets into male diabetic NOD SCID mice (15-25 weeks old) has been implemented by transplantation centers to assess islet function to reverse diabetes induced by intraperitoneal injection of 50 mg/kg streptozotocin (STZ; Sigma-Aldrich) for three consecutive days. NOD SCID mice with hyperglycemia (>350 mg/dL) for at least two consecutive days were transplanted with identical numbers (1200 IEQ) of single donor human islets under the left kidney capsule. Blood glucose was determined 2-3 times per week for 28 days using a glucometer (LifeScan Inc.). Transplant recipients that had two consecutive blood glucose measures <200 mg/dl were considered to have reversed diabetes and the first date of blood glucose measure <200mg/dl was considered date of diabetes reversal. The reversal rate of diabetes was calculated based on the ratio of number of mice that reversed diabetes to that of total number of mouse transplanted in each group.

Results

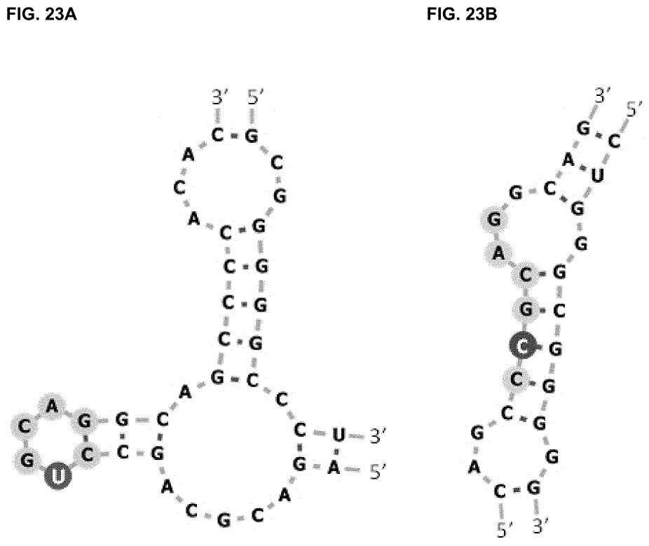

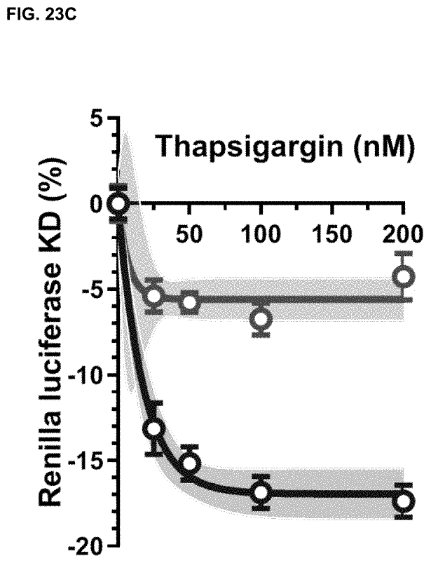

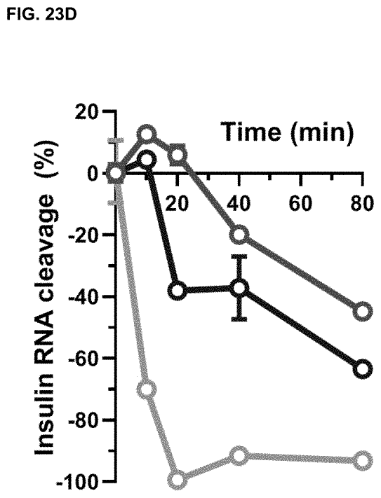

Protective insulin mRNA can be cleaved by IRE1α and decays more rapidly during ER stress. The INS 3′ UTR contains two SNPs (rs3842752 and rs3842753) generating variation of insulin mRNA, the protective (U-A) and susceptible (C-C) variant. The secondary structure of both mRNA variants was derived from in-silico modelling [mFOLD ref]. Protective insulin mRNA contains the CUGCAG-motif and is able to form the hairpin structure required for RIDD, whereas susceptible insulin mRNA does neither (FIGS. 23A-23B), implying that only the protective insulin mRNA variant fulfills both prerequisites to be recognized and cleaved by IRE1α.

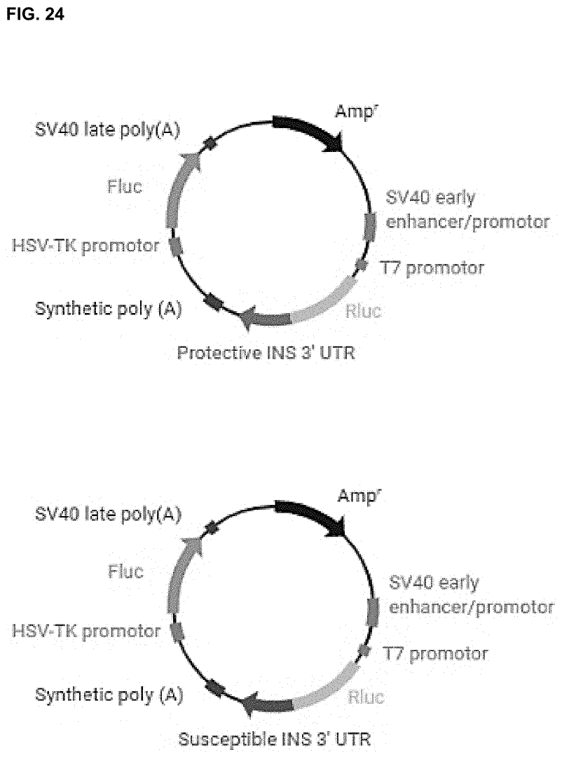

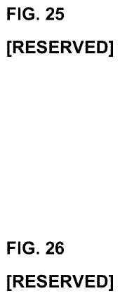

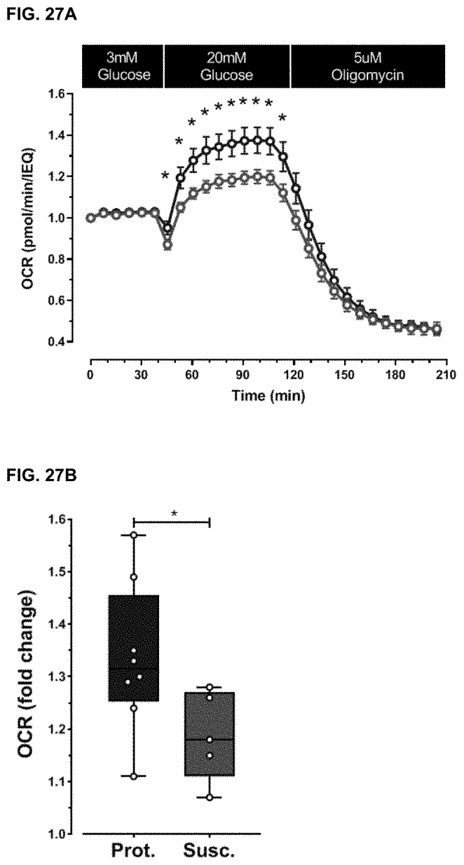

Next, the effect of INS variation on mRNA stability during ER stress was determined using a dual luciferase reporter assay. The INS 3′ UTR was cloned downstream of renilla luciferase (Rluc) to generate two vectors either carrying protective Rluc-INS or susceptible Rluc-INS (FIG. 24). These vectors were transfected into HEK293T cells and ER stress was induced with increasing amounts of thapsigargin (TG), followed by luciferase activity measurement. The decline in Rluc activity was significantly faster in cells transfected with protective Rluc-INS compared to susceptible Rluc-INS (FIG. 23C), implying that the protective INS 3′ UTR is less stable during ER stress.

To determine whether decay of insulin mRNA is IRE1α dependent, human insulin mRNA was cleaved in vitro by activated IRE1a. Total mRNA isolated from heterozygous protective (n=9), homozygous protective (n=1) and homozygous susceptible islet donors (n=8) was incubated with recombinant phosphorylated IRE1α, followed by insulin-specific RT-qPCR. Protective insulin mRNA from donors homozygous and heterozygous for the protective INS variant was cleaved significantly more compared to susceptible insulin mRNA (FIG. 23D). These findings reveal that IRE1α can cleave human insulin mRNA, and that cleavage of protective insulin mRNA is increased and more rapid compared to susceptible insulin mRNA. RIDD of the protective insulin mRNA variant could therefore reduce translational burden during ER stress, and suggests that ER stress can be resolved more easily in beta cells carrying the protective INS variant.

Reduced ER stress and immunogenicity in islets with protective INS variant. While the INS variation lies in the UTR of the canonical reading frame, it leads to translation in the +2 ribosomal reading frame coding for INS-DRiP. It was previously reported that the N-terminus is naturally processed by both beta-cells and antigen-presenting cells (APC), leading to presentation by HLA class I and class II, respectively. CD8 T-cells of T1D recognizing the invariant N-terminus destroyed human beta-cells in and stress and inflammation dependent fashion. Proliferative T-cells (predominantly CD4) responding APC pulsed with to recombinant INS-DRiP in T1D patient carrying the susceptible INS variant cross-reacted with both the susceptible and protective INS-DRiP variant, confirming that the epitope is unlikely covering the INS-DRiP variant region.

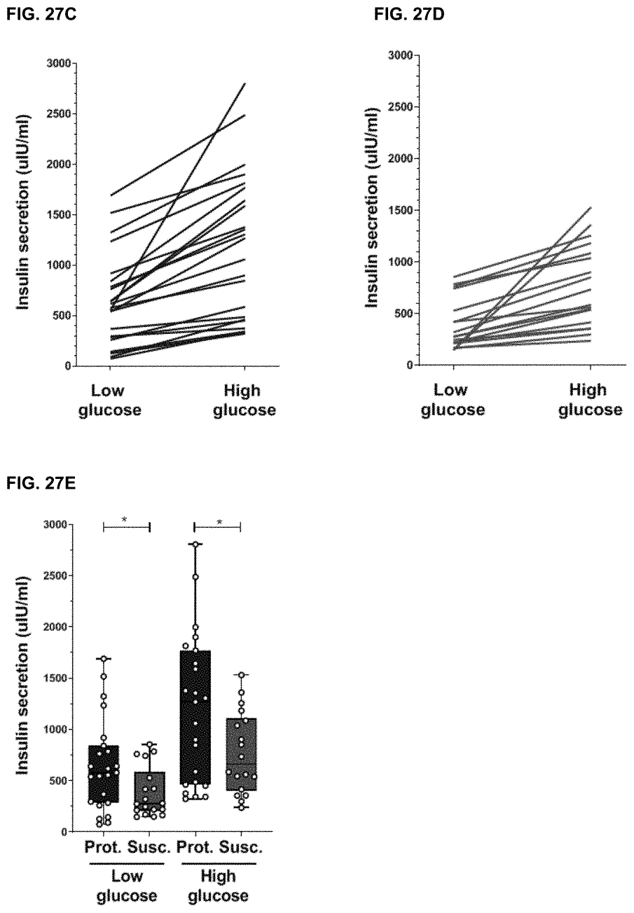

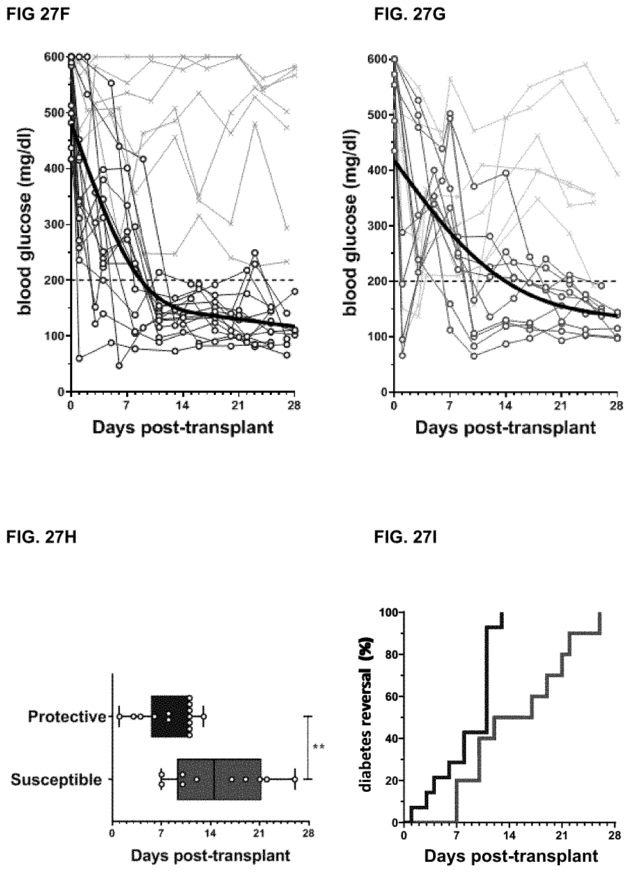

Improved vitality and function of human islets carrying the protective INS variant. Ex vivo function of islets was determined by measuring oxygen consumption rate (OCR) and insulin secretion. Islets with the protective INS variant had a significantly higher OCR and OCR fold change compared to islets carrying the homozygous susceptible INS variant (FIGS. 27A-B), indicative of greater activity. Furthermore, islets with the protective INS variant secreted more insulin in low and high glucose conditions compared to islets with the susceptible INS variant (FIGS. 27C-E), with similar stimulation indexes. Thus, islets with the protective INS variant have a better metabolic function and secrete more insulin in response to glucose, suggesting increased capability to respond to energetic demand (such as high glucose levels) and regulate blood glucose levels.

To evaluate in vivo beta cell function, human islets were transplanted into NOD SCID mice made diabetic by streptozotocin (STZ) treatment, and blood glucose levels were monitored for four weeks post-transplantation. Animals that reversed diabetes became normoglycemic twice as fast when they were transplanted with islets carrying the protective INS variant compared to the susceptible variant only (diabetes remission within 13 vs 26 days, respectively; FIGS. 27F-I). Diabetes reversal rate was similar between the protective and susceptible INS variant (14 out of 21 (67%) vs 10 out of 16 (63%), respectively). These results point to improved in vivo function of islets with the protective INS variant. Homozygosity for protective INS is rare (±2%). Fortunately, one pancreas donor proved homozygous protected. Although anecdotal, islets from this donor showed consistently higher rates of vitality and function. Insulin mRNA from the homozygous protective donor was cleaved more and faster than that of heterozygous protective donors (cleavage within 20 minutes >90% vs <40%, respectively; FIG. 23D). Additionally, islets from the homozygous protective donor had the highest OCR fold change (1.57 pmol/min/IEQ; FIG. 23B), and the highest insulin secretion in both low and high glucose conditions (mean of 1510 and 2129 μlU/ml, FIGS. 27C & 27E), reversing diabetes in mice in vivo within 4 to 8 days (FIGS. 27F-I). These results suggest a gene dosing effect of the protective insulin allele, in which homozygous protective islets function superior to heterozygous protective islets, which is consistent with increased RIDD of insulin mRNA.

Discussion

The results above indicate that the protective INS SNP variant has several clinically significant benefits and advantages in T1D patients. First, the protective SNP reduces autoimmunity against islet cells-likely due to a reduction in insulin antibody production against INS-DRiP antigens, thereby preventing or reducing damage to islets. Second, the protective SNP preserves beta cell function, which translates to improved glycemic control in T1D patients. Third, the protective SNP may prevent or reduce diabetic complications in T1D patients with protective INS variant. Analysis of a diabetic complication database (DCCT/EDIC) may lead to a way to predict whether T1D patients with the protective SNP are less susceptible to developing complications, and may lead to improvement in personalized medicine for those patients at less risk. For example, T1D patients having the protective INS variant may have less need for statin therapy of may need lower or fewer doses of insulin to maintain stable glycemic control.

The results (see, e.g., FIG. 23C) may also explain the lower mRNA expression of protective INS allele in the pancreas in accordance with the central tolerance mechanism discussed herein. Protection from T1D inferred by INS variation (IDDM2) has previously been attributed to central tolerance, in which class III VNTR alleles increased thymic expression of insulin mRNA. Due to high LD (r²=1) between the VNTR and polymorphisms in the INS haplotype it is possible that these polymorphisms are etiological as well. Here, an explanation for genetic protection from T1D is inferred by polymorphisms in the INS 3′ UTR (rs3842752 and rs3842753; AKA +1127Pstl and +1140A/C respectively) involving peripheral tolerance, which may act in concert with the previously proposed mechanism of central tolerance. Lower pancreatic expression of the protective INS allele is likely because of breakdown by IRE1α and not because of VNTR influencing insulin mRNA expression. Unfolded protein response (UPR) and IRE1α are always activated in beta cells due to high protein folding demand leading to cleavage of protective insulin mRNA by IRE1α.

The results above suggest that in beta cell replacement therapy, islets or stem cell-derived beta cells (e.g., beta cells derived from induced pluripotent stem cells (iPSC) or embryonic stem cells (ePSC)) having protective INS gene may function better, and only those cells with the protective INS gene should be used as donors for transplantation to better ensure successful reversal, prevention, or treatment of T1D.

RIDD of mRNA can reduce translational burden in stressed beta cells. It is shown that IRE1α cleaves human insulin mRNA (which can make up to 20% of the beta cell transcriptome; 100.000-200.000 molecules per beta cell) with protective polymorphisms more and more rapidly compared to insulin mRNA with susceptible polymorphisms. Beta cells carrying the protective INS variant (i) have better cellular function, (ii) secrete more insulin, (iii) reverse diabetes more rapidly in mice; and are likely (a) experience less stress and (b) are less visible to immune surveillance compared to beta cells with susceptible INS variant.

Carrying the protective INS variant has been associated with lower incidence of insulin autoantibodies in T1D patients and their offspring, and more importantly higher C-peptide levels and lower HbA1c in T1D patients, which could be explained by the mechanism of peripheral tolerance. Since lower HbA1c suggests lower risk for diabetes complications the DCCT cohort can be analyzed for frequency of complications in T1D patients with long term disease.

This method of RIDD of human insulin mRNA provides an alternative explanation for the lower levels of the protective insulin mRNA variant found in the pancreas and provides an explanation for the previously suggested beta cell-specific effect of INS variation. Additionally, INS expression in beta cells might not be allele-specific, but an artifact of preferential RIDD of the protective insulin mRNA variant.

Genotyping the INS 3′ UTR can help predict function and vitality of human islets isolated for transplantation and it might be helpful to derive beta cells or pseudo islets from stem cells that harbor these T1D-protective genetic traits.

### Example 5

### Inverse Link Between T1D and Cancer (Insulinoma)

Type 1 diabetes (T1D) is a disease that results in insufficient insulin production. Insulinoma is a cancer that results in too much insulin production. These studies are undertaken to understand whether there is an inverse relationship between the two diseases by exploring differences in T1D-associated genes. The immune system has to be in perfect balance to function optimally. It has to react adequately to infections and cancerous cells without causing autoimmune problems. During autoimmune disease the immune response is too strong, leading to killing of beta cells by autoreactive T cells, whereas during insulinoma the immune response is too weak giving cancerous beta cells a change to proliferate out of control. Taking a closer look at the beta cell, one point of view is that beta cells can make too many autoantigens and attract immune cells during stress, to which the immune system responds ‘with good intentions’: by removing dysfunctional beta cells. In contrast, beta cells make too few antigens and hide from immune cells in case of insulinoma, to which the immune system cannot respond, allowing the cancerous cells to proliferate. Due to these opposing effects, an objective of this research is to identify if there are inverse genetic relationships between type 1 diabetes (T1D) and cancer.

In these studies, the presence of new or established T1D-associated gene variants may be determined in insulinoma patients. Specifically, the frequency of candidate SNPs (e.g., rs3842752 and rs3842753) can be compared in those with T1D or insulinoma and quantification of differences at the transcriptome level may also be evaluated. Study datasets are obtained from the NCBI database of genotypes and phenotypes (dbGaP). Due to the heterogeneous definition of case, each cohort is analyzed separately.

In addition, as discussed above, it was found that beta cells can make INS-DRiP during ER stress and that autoreactive T cells can respond INS-DRiP, showing INS-DRiPs role in T1D pathogenesis. Since DRiP's have also been associated with several cancers these studies are designed to investigate whether INS-DRiP protein expression can be detected in insulinomas, whether T cells that respond to islet antigens (INS-DRiP) can be found in blood from insulinoma patients, and whether primary insulinoma cell lines can be killed with an INS-DRiP-specific T cell clone.

Moreover, because it was recently discovered that INS gene variation may impact the production of INS-DRiP in beta cells, beta cell (dys-)function, and antigen presentation, these studies are designed to investigate the link between INS gene variation and insulinomas, and then to compare those links to T1D. Specifically, these studies are designed to determine:
(i) Whether the frequency in INS gene variant (protective or susceptible-for T1D) is different in insulinoma patients compared to the population or T1D patients. In particular, given the likelihood that most T1D patients have the susceptible insulin variant, the studies may determine whether insulinoma patients have the protective variant since beta cells with the protective INS variant make less INS-DRiP and are less immunogenic.
(ii) Whether insulinoma patients with a protective or susceptible INS variant have a better or worse prognosis.
(iii) Whether ER stress status is different in patients with either INS variant given that neoantigens (INS-DRiP) in cancer and T1D can be produced during ER stress.
(iv) Whether immune markers are different between INS variants.
(v) Whether there is a difference in insulin (or insulin-SPLICE) expression between patients with either insulin variant and whether such a difference correlates with hypoglycemia in insulinoma patients.

To determine the answer to these inquiries, specific information from insulinoma patients can be gathered and analyzed, such as whether the insulinoma is benign or malignant, whether the patient had any response to therapy (and what type of therapy was used), whether the patient experienced hypoglycemia, and the patient's HbA1C levels.

Further, using an NIH repository of insulinoma samples, DNA sequencing may be used to determine VNTR class and presence (or absence) of SNPs (r53842752 and rs3842753) in the INS gene or whole INS haplotype, as well as the HLA type. RNA sequencing may also be used to determine the expression of insulin, insulin-SPLICE junction, and one or more immune markers and/or ER stress markers (e.g., TAP, Transglutaminase (TG), Peptidyl arginine deiminase (PAD), HLA class I, B2-microglobulin, CXCL10, STAT1, NfkB, IRF1, JNK, IRE1a, ATF6, PERK, XBP1, CHOP, BiP/GPR78, ATF4, eIF2).

### Example 6

### Protective Variant Confers Reduced Immunogenicity

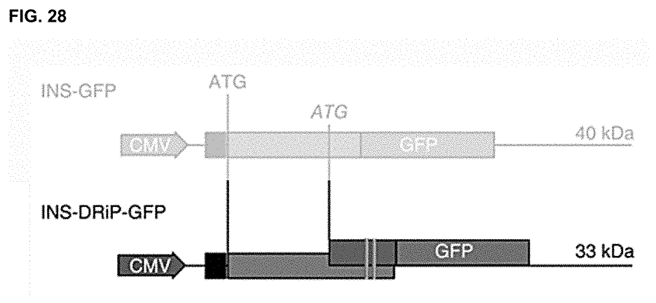

Method: Two vectors of INS-DRiP-GFP (one of the protective genotype and one of the susceptible genotype) that express insulin and DRiP linked to GFP were created (FIG. 28). The vectors were transfected into cells, and GFP expression was observed when translation is started at the DRiP start codon.

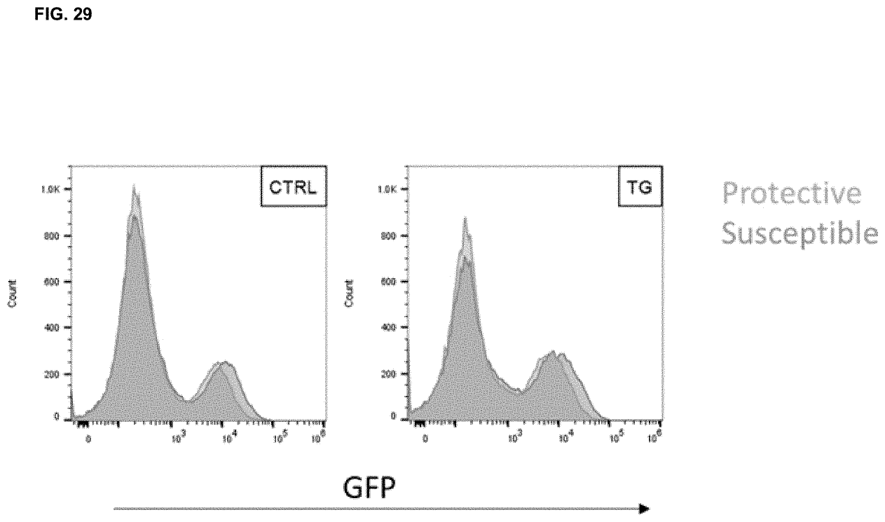

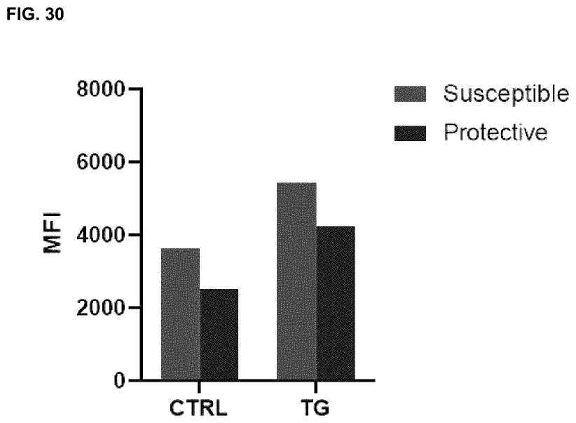

Result: The protective variant (blue) shows lower DRiP-GFP expression compared to the susceptible variant (FIG. 29, left panel; FIG. 30, CTRL). During ER stress (TG treatment) the expression of DRiP-GFP is increased in both variants, and the DRiP-GFP expression in the protective variant is still lower than the susceptible variant (FIG. 29, right panel; FIG. 30, TG).

Conclusion: Cells transfected with the protective construct make less DRiP-GFP. Since DRiP is a neoantigen, cells harboring the protective variant are likely less immunogenic compared to susceptible variant.

1. A targeting molecule for inhibiting defective ribosomal product (DRiP) expressioncomprising:
(a) a donor template comprising a nucleic acid sequence that encodes an mRNA sequence comprising CUGCAG to replace a single nucleotide polymorphism in the 3′ untranslated region of the insulin (INS) gene;
(b) a nucleic acid sequence that is fully or partially complementary to at least a portion of INS mRNA; or an antibody or fragment thereof that targets; wherein the targeting molecule inhibits the expression of DRiP; or
(c) an antibody or fragment thereof that binds to DRiP, a peptide that binds to DRiP, or a nucleic acid that binds to DRiP; wherein binding of the targeting molecule prevents MHC presentation of a DRiP antigen.

2. (canceled)

3. (canceled)

4. The targeting molecule of claim 1, wherein the single nucleotide polymorphism is rs3842752.

5. The targeting molecule of claim 1, wherein the nucleic acid sequence of (b) is an siRNA or an shRNA.

6. (canceled)

7. The targeting molecule of claim 1, wherein the nucleic acid sequence of (a) or (b) is an antisense oligo.

8. The targeting molecule of claim 1, wherein the nucleic acid sequence of (a) or (b) is an RNA or cDNA molecule.

9. The targeting molecule of claim 1, wherein the nucleic acid sequence that is fully or partially complementary to at least a portion of INS mRNA comprises a nucleic acid sequence beginning at AUG341 in the INS gene.

10. The targeting molecule of claim 1, wherein the nucleic acid sequence that is fully or partially complementary to at least a portion of INS mRNA comprises a nucleic acid sequence beginning at AUG60 in the INS gene.

11. The targeting molecule of claim 1, wherein the nucleic acid that binds to DRiP is an aptamer.

12. A pharmaceutical composition comprising the targeting molecule of claim 1 and one or more pharmaceutically acceptable carriers or excipients.

13. The pharmaceutical composition of claim 12, further comprising one or more adjuvants.

14. The pharmaceutical composition of claim 12, further comprising one or more additives.

15. The pharmaceutical composition of claim 13, further comprising one or more drugs.

16. A method for preventing or treating type 1 diabetes (T1D) comprising administering to a subject a therapeutically effective amount of the targeting molecule of claim 1, wherein the subject is identified as having a susceptible SNP mutation or lacking a protective SNP mutation.

17. A method for inhibiting the expression or activity of defective ribosomal products (DRiPs) in pancreatic beta cells comprising contacting the pancreatic beta cells with an effective amount of the targeting molecule of claim 1, wherein the pancreatic beta cells have a susceptible SNP mutation or lack a protective SNP mutation.

18. A method for preventing or treating type 1 diabetes (T1D) comprising transplanting a therapeutically effective amount of donor beta cells to a subject having or at risk of developing T1D, wherein the donor beta cells carry a protective insulin gene SNP.

19. The method of claim 18, wherein the donor beta cells are stem cells or islet cells from an allogenic donor.

20. The method of claim 18, wherein the donor beta cells are stem cells or islet cells obtained from the subject having or at risk of developing T1D prior to the transplanting step.

21. The method of claim 18, wherein the donor cells are selected from stem cells or islet cells that genetically screened and determined to carry the protective insulin gene SNP.

22. The method of claim 18, wherein the donor cells are modified by contacting the donor cells with a targeting molecule to (i) delete or knock-out the AUG341 of an insulin gene, (ii) replace a single nucleotide polymorphism in the 3′ untranslated region of the insulin (INS) gene to carry the protective insulin gene SNP, or (iii) both (i) and (ii).


SEQUENCE LISTING (24 sequences)

SEQ ID NO: 1 - RNA, 6 nt, Artificial Sequence
  consensus sequence
cugcag

SEQ ID NO: 2 - protein, 9 aa, Artificial Sequence
  INS-DRiP1-9 epitope
MLYQHLLPL

SEQ ID NO: 3 - RNA, 468 nt, Homo sapiens
  misc_feature (1)..(63): 5′ and 3′ untranslated region
  misc_feature (60)..(62): AUG codon
  misc_feature (64)..(468): full-length insulin mRNA with bona fide PPI ORF
  misc_feature (72)..(74): AUG codon
  misc_feature (107)..(109): putative non-AUG (CUG) start site upstream
  misc_feature (341)..(343): AUG codon
  misc_feature (399)..(399): uracil or cytosine
  misc_feature (413)..(413): adenine or cytosine
agcccuccaggacaggcugcaucagaagaggccaucaagcagaucacuguccuucugccauggcccuguggaugcgccuccugccccugcuggcgcugcuggcccucuggggaccugacccagccgcagccuuugugaaccaacaccugugcggcucacaccugguggaagcucucuaccuagugugcggggaacgaggcuucuucuacacacccaagacccgccgggaggcagaggaccugcagguggggcagguggagcugggcgggggcccuggugcaggcagccugcagcccuuggcccuggaggggucccugcagaagcguggcauuguggaacaaugcuguaccagcaucugcucccucuaccagcuggagaacuacugcaacuagacgcagcngcaggcagccccncacccgccgccuccugcaccgagagagauggaauaaagcccuugaaccagcaaaa

SEQ ID NO: 4 - protein, 110 aa, Homo sapiens
  MISC_FEATURE (95)..(95): AUG codon
  MISC_FEATURE (95)..(95)
MALWMRLLPLLALLALWGPDPAAAFVNQHLCGSHLVEALYLVGGERGFFYTPKTRREAEDLQVGQVELGGGPGAGSLQPLALEGSLQKRGIVEQCCTSICSLYQLENYCN

SEQ ID NO: 5 - protein, 43 aa, Homo sapiens
  MISC_FEATURE (21)..(21): cysteine or arginine
  MISC_FEATURE (25)..(25): histidine or proline
MLYQHLLPLPAGELLQLDAACRQPHTRRLLHRERWNKALEPAK

SEQ ID NO: 6 - RNA, 13 nt, Artificial Sequence
  hairpin structure
ucucugcagaaga

SEQ ID NO: 7 - RNA, 13 nt, Artificial Sequence
  hairpin structure
gggcugccguccc

SEQ ID NO: 8 - RNA, 16 nt, Artificial Sequence
  hairpin structure
gcgugggcugcagcgc

SEQ ID NO: 9 - RNA, 15 nt, Artificial Sequence
  hairpin structure
acaccugcagagugu

SEQ ID NO: 10 - RNA, 15 nt, Artificial Sequence
  hairpin structure
gacccagcagugguc

SEQ ID NO: 11 - RNA, 16 nt, Artificial Sequence
  hairpin structure
uggacugcaguuucua

SEQ ID NO: 12 - RNA, 16 nt, Artificial Sequence
  hairpin structure
aguucugcaagugacu

SEQ ID NO: 13 - RNA, 15 nt, Artificial Sequence
  hairpin structure
ggcccugcaguggcc

SEQ ID NO: 14 - RNA, 19 nt, Artificial Sequence
  hairpin structure
gagguccugcagcgcuuuc

SEQ ID NO: 15 - RNA, 6 nt, Artificial Sequence
  stemloop cleavage insulin variant
ccgcag

SEQ ID NO: 16 - RNA, 27 nt, Artificial Sequence
  motif
agacgcagccugcaggcagccccacac

SEQ ID NO: 17 - RNA, 11 nt, Artificial Sequence
  hairpin structure
gcgggggcccu

SEQ ID NO: 18 - RNA, 14 nt, Artificial Sequence
  motif without hairpin structure
cagcccgcaggcag

SEQ ID NO: 19 - RNA, 11 nt, Artificial Sequence
  motif without hairpin structure
cugggcggggg

SEQ ID NO: 20 - DNA, 19 bp, Artificial Sequence
  forward primer
ggaggggctcacaacagtg

SEQ ID NO: 21 - DNA, 20 bp, Artificial Sequence
  reverse primer
gtgtctccctgactgtgtcc

SEQ ID NO: 22 - DNA, 23 bp, Artificial Sequence
  forward primer
ctggagaactactgcaactagac

SEQ ID NO: 23 - DNA, 20 bp, Artificial Sequence
  reverse primer
tgctggttcaagggctttat

SEQ ID NO: 24 - DNA, 22 bp, Artificial Sequence
  probe
ctcctgcaccgagagagatgga